(19) United States
(12) Patent Application Publication
(10) Pub. No.: US 2025/0367164 A1
(43) Pub. Date: Dec. 4, 2025

(54) THERAPEUTIC COMPOSITIONS FOR SKIN DISORDERS AND WOUND REPAIR

(71) Applicant: UNIVERSITY OF MIAMI, Miami, FL (US)

(72) Inventors: Ivan JOZIC, Miami, FL (US); Marjana TOMIC, Miami, FL (US); Fotios M. ANDREOPOULOS, Miami, FL (US)

(21) Appl. No.: 18/861,068

(22) PCT Filed: Apr. 27, 2023
(86) PCT No.: PCT/US2023/020173

§ 371 Date: Oct. 28, 2024

Related U.S. Application Data
(60) Provisional application No. 63/335,306, filed on Apr. 27, 2022.

(51) Int. Cl. A61K 31/366; A61K 9/06; A61K 45/06; A61K 47/40; A61K 47/42; A61L 26/00; A61P 17/02; A61P 31/04

(52) U.S. Cl. CPC A61K 31/366; A61K 9/06; A61K 45/06; A61K 47/40; A61K 47/42; A61L 26/0023; A61L 26/0028; A61L 26/0066; A61L 26/008; A61P 17/02; A61P 31/04

(57) ABSTRACT

Disclosed herein are therapeutics compositions including one or more active agents, for instance a statin, cyclodextrin, or combination thereof. The compositions are useful in the treatment of tissue injuries, including wounds, as well as skin inflammation and infections.

### CROSS-REFERENCE TO RELATED APPLICATION

This application claims the benefit of U.S. Provisional Application 63/335,306, filed Apr. 27, 2022, the contents of which are hereby incorporated in its entirety.

### FIELD OF THE INVENTION

The invention is directed to therapeutics compositions including one or more active agents, for instance a statin, cyclodextrin, or combination thereof. The compositions are useful in the treatment of tissue injuries, including chronic wounds, inflammatory disorders as well as infections.

### BACKGROUND

Over eight million Americans suffer from chronic wounds (e.g., diabetic foot, venous leg and pressure ulcers) annually and this number is likely to rise with the aging population and the increasing incidence of obesity and diabetes. Lack of understanding regarding the molecular mechanisms underpinning impaired healing in chronic wounds leads to increased mortality and serious co-morbidities including frequent lower leg amputations. In addition, impaired re-epithelialization is a well-recognized contributing factor to chronic wounds. Diabetic foot ulcers (“DFUs”) represent a significant burden to patients, health care professionals, and the US health care system, affecting up to 34% of persons with diabetes mellitus and resulting in an approximate yearly cost of $58 billion annually in the US alone. Healing results in remission, not cure, as up to 2 of every 3 ulcers re-occur within 5 years. Thus, in order to reduce excessive limb amputation and the high mortality resulting from DFUs, effective treatment strategies need to both promote wound closure as well as inhibit wound infection. Venous leg ulcers (VLUs) develop as a consequence of venous hypertension caused often by venous valvular incompetence leading to venous reflux or venous obstruction, impacting over 2 million people annually. Unfortunately, with nearly a third of the patients require >2 years to heal with a recurrence rate of 60-70% and mortality rates ranging from 5-19%. The presence of an unhealed wound significantly increases the patient's risk of infection and additional complications such as pain, mobility, osteomyelitis, gangrene, loss limb, other systemic complications and even loss of life. Their chronicity, frequent relapses, and associated complications heavily impact patient's quality of life. Prolonged inflammation in wounds profoundly hinders epithelial closure, granulation tissue formation and delays wound healing. Furthermore, recent data show that inflammation in chronic wounds, although persistent, is ineffective to trigger acute wound response and progression of healing.

Wound healing is a complex multi-factorial process that requires a well-coordinated cellular program involving multiple cell types and cellular processes aiming for barrier restoration and maintenance that requires tight spatial and temporal control. Keratinocytes, fibroblasts, endothelial cells, local and circulating immune and progenitor cells are all involved to properly execute inflammation, epithelialization, granulation tissue formation, angiogenesis and matrix deposition and reorganization. Thus, any de-regulation of cellular function can lead to major clinical impact. In order to target multiple cellular processes to promote healing, the treatment approach needs to be multifactorial, i.e. to target multiple cellular processes.

Cyclodextrins are a family of cyclic oligosaccharides that are commonly used as molecular chelating agents. They are able to form inclusion complexes with a wide variety of hydrophobic molecules by virtue of having a relatively hydrophilic outer surface that can dissolve in water and a relatively hydrophobic cavity which provide a hydrophobic matrix to capture appropriately sized non-polar moieties. These complexes can release pre-loaded molecules over prolonged periods of time. Hence, they have been explored in pharmaceutical formulations to improve the solubility of substances and enhance drug delivery through biological membranes, protection against degradation by microorganisms, masking of malodors and bitter tastes, as well as protection against UV damage. They are absorbed in very low quantities in the upper intestinal tract and metabolized only by the bacteria in caecum and colon. Furthermore, due to their ability to bind cholesterol and free fatty acids, cyclodextrins (methyl-β-cyclodextrin (MβCD) and hydroxypropyl-β-cyclodextrin (HβCD), in particular) have been widely used in extracting cholesterol from plasma membranes. Cholesterol is an essential part of every mammalian cellular and organelle membrane; it is involved in various cellular processes ranging from maintaining membrane fluidity/permeability, membrane trafficking, cell signaling, protein/lipid sorting as well as formation of ordered liquid domains in membranes such as caveolae (Ω-shaped cell membrane invaginations) and lipid rafts. Moreover, cholesterol is a precursor to all steroids and skin has recently been demonstrated to be an extra-adrenal site of cortisol production, via local synthesis of all elements of the classical HPA axis. Interestingly, chronic wounds exhibit elevated levels of cortisol, thus introducing cholesterol as a viable therapeutic target for treatment of chronic wounds.

One of the hallmarks of non-healing wounds is loss of keratinocyte migration, often in the presence of well-developed granulation tissue. However, no current treatments target this process. Caveolin-1 (Cav1) is a principal component of caveolae which inserts itself into areas of the cell membrane that are high cholesterol and sphingomyelin and functions to recruit subsequent molecules for assembly of caveolae. Cav1 is upregulated in non-healing chronic wounds and serves to inhibit directional keratinocyte migration necessary for proper wound closure. Binding of Cav1 to various growth factor receptors (EGFR, VEGFR, PDGFR, etc.) usually results in their antagonism. Thus, it is not surprising that all but one growth factor-based therapy failed to achieve efficacy by the FDA for treatment of DFUs or VLUs (the last FDA approved biologic was rh-PDGF-BB in 2005), since upregulation of Cav1 would sequester and antagonize their cognate growth factor receptors and thus make growth factor-based therapies futile for treatment of DFUs. Interestingly, cortisol induces expression of Cav1 and acts to inhibit directional cell migration necessary for wound re-epithelialization by perturbing actin-cytoskeletal dynamics. Thus, spatiotemporal downregulation of Cav1 (either by CRISPR-mediated knockdown or cholesterol depletion via statins and cyclodextrins) can ameliorate the inhibitory effects of Cav1 on keratinocyte migration and subsequent wound closure. Moreover, it is well known that wounds are colonized by opportunistic pathogens, with S. aureus and P. aeruginosa being most common bacteria isolated from chronic wounds. Interestingly, P. aeruginosa internalization requires interaction with host Cav1 protein, which upon entry can evade lysosomal degradation and persist inside the host cells by secreting various virulence factors that disrupt endomembrane trafficking. However, it is yet to be determined whether the same is true for S. aureus.

There remains a need for improved compositions and methods for the treatment of wounds, including chronic wounds. There remains a need for synergistic combinations of agents that promote wound closure through multiple mechanisms, including promote wound closure by utilizing a multi-pronged approach that aims to ameliorate: 1) promoting cell migration and wound re-epithelialization, 2) promoting angiogenesis, and 3) inhibiting intracellular pathogen colonization, in the hopes of converting a non-healing chronic wound into an acute healing wound.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1A depicts a dressing having a first layer that is continuous with a backing layer (side view).

FIGS. 1B and 1C depict a dressing having a first layer that is discontinuous with a backing layer (FIG. 1B depicts a side view; FIG. 1C depicts a top view).

FIG. 2A depicts a dressing having a first layer, first intermediate layer, and backing layer, in which the first layer is continuous with the intermediate layer, and the intermediate layer is continuous with the backing layer.

FIG. 2B depicts a dressing having a first layer, first intermediate layer, and backing layer, in which the first layer is discontinuous with the intermediate layer, and the intermediate layer is continuous with the backing layer.

FIG. 2C depicts a dressing having a first layer, first intermediate layer, and backing layer, in which the first layer is discontinuous with the intermediate layer, and the intermediate layer is discontinuous with the backing layer.

FIG. 3A depicts a dressing having a first layer, second layer, and backing layer, in which the second layer is continuous with the first layer, and the first layer is continuous with the backing layer.

FIG. 3B depicts a dressing having a first layer, second layer, and backing layer, in which the second layer is discontinuous with the first layer, and the first layer is continuous with the backing layer.

FIG. 3C depicts a dressing having an intermediate layer disposed between the first and second layer, in which the second layer is continuous with the intermediate layer, the intermediate layer is continuous with the first layer, and the first layer is continuous with the backing layer.

FIG. 3D depicts a dressing having a second intermediate layer disposed between the first and second layer, in which the second layer is continuous with the second intermediate layer, the second intermediate layer is continuous with the first layer, a first intermediate layer disposed between the first layer and the backing layer, the first intermediate layer is continuous with the first layer and the first intermediate layer is continuous with the backing layer.

FIG. 4A depicts a dressing having a first layer adjacent to the second layer, wherein each of the first and second layers have a backing layer that is continuous with the first and second layers. In certain implementations (not shown) the dressing has a first layer adjacent to a second layer, and a single backing layer. When no intermediate layers are present, the backing layer is discontinuous with respect to either the first layer or second layer individually, and the backing layer may be continuous or discontinuous to the first and second layers taken together.

FIG. 4B depicts a dressing having a first layer adjacent to the second layer, where there is a single intermediate layer disposed between the backing layer and each of the first and second layers.

FIG. 4C depicts a dressing having a first layer adjacent to the second layer, where there is a separate intermediate layer disposed between each of the first and second layers and the backing layer.

FIG. 5A depicts a dressing having a wound contacting layer, first layer, second layer, and backing layer, in which the wound contacting layer is continuous with the second layer, the second layer is continuous with the first layer, and the first layer is continuous with the backing layer.

FIG. 5B depicts a dressing according to FIG. 4C, further having a wound contacting layer that is continuous with the first layer and the second layer, taken together.

FIG. 5C depicts a dressing according to FIG. 4A, further having a wound contacting layer that is continuous with the first layer and the second layer, taken together.

FIG. 5D depicts a dressing according to FIG. 3D, further having a wound contacting layer that is continuous with the second layer.

FIG. 5E depicts a dressing having a first wound contacting layer than is continuous with the first layer, and a second wound contacting layer that is continuous with the second layer, wherein the first and second layers are adjacent to each other, and continuous with a first backing layer, and second backing layer, respectively.

FIG. 5F depicts a dressing having a first wound contacting layer than is continuous with the first layer, and a second wound contacting layer that is continuous with the second layer, wherein the first and second layers are adjacent to each other, a first intermediate layer and a second intermediate layer, wherein the first intermediate layer is continuous with a first backing layer, and the second intermediate layer is continuous with a second backing layer.

FIG. 6A depicts cyclodextrin cytotoxicity are various concentrations in primary human karatinocytes.

FIG. 6B depicts cyclodextrin cytotoxicity are various concentrations in primary human karatinocytes.

FIG. 7 depicts cytotoxicity of statins in combinations with cyclodextrins; statins alone (FIG. 7A), Hβ-CD+lovastatin (FIG. 7B), Hβ-CD+mevastatin (FIG. 7C), Hβ-CD+simvastatin (FIG. 7D).

FIG. 8A depicts cytotoxicity of cyclodextrins combined with 2 statins; FIG. 8B depicts cytotoxicity of cyclodextrins combined with 3 statins.

FIG. 9A depicts delivery of statins to skin and demonstrates cholesterol disruption by cyclodextrin-statin hydrogel.

FIG. 9B depicts delivery of statins to skin and tissues that were harvested, cryosectioned and traceable fragments of statin powder (C₅H₁₀O and C₄H₈O₃) visualized by mass spectrometry.

FIG. 9C depicts delivery of statins to skin and demonstrates cholesterol disruption by cyclodextrin-statin hydrogel with levels of cholesterol sulfate determined by time-of-flight secondary ion mass spectrometry.

FIG. 10 depicts images showing wound closure ability of cyclodextrin-statin hydrogels.

FIG. 11 depicts histological quantification of wound closure in splinted db/db mice treated with cyclodextrin-statin hydrogels.

FIG. 12 depicts quantification of splinted db/db mouse wounds treated with cyclodextrin-PDGF.

FIG. 13 depicts upregulation of angiogenic markers in db/db mouse wounds treated with cyclodextrin-statin hydrogels.

FIG. 14 depicts the validation of elevated production of angiogenic marker VEGF by ELISA.

FIG. 15 depicts macrophage polarization towards M2-like by cyclodextrin/statin hydrogel.

FIG. 16 depicts inhibition of intracellular MRSA by cyclodextrin treatment.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

A group is optionally substituted unless expressly provided otherwise. The term “optionally substituted” refers to being substituted or unsubstituted. In certain embodiments, alkyl groups are optionally substituted. “Optionally substituted” refers to a group which may be substituted or unsubstituted (e.g., “substituted” or “unsubstituted” alkyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

As used herein, the term hydroxyproline refers to the compound (2S,4R)-4-hydroxypyrrolidine-2-carboxylic acid.

As used herein, a “target functional group,” when used in the context of crosslinking reaction or crosslinked gel, refers to the functional groups within a polypeptide that chemically react with a given crosslinking reagent. For example, carbodiimide crosslinkers react with carboxylic acid functional groups, and aldehyde-based crosslinkers react with amino functional groups.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are compositions that include a cyclodextrin and a hydrogel. The cyclodextrin may be dispersed, dissolved, or otherwise suspended in the hydrogel. The composition can further include at one or more active agents—in some embodiments the active agent is complexed with the cyclodextrin, while in other embodiments the active agent is not complexed with the cyclodextrin. Unless specified to the contrary, designation of an active agent as complexed with the cyclodextrin is not intended to exclude the presence of small amounts of active agent that is not complexed with the cyclodextrin. Similarly, unless specified to the contrary, the designation of an active agent as not-complexed with a cyclodextrin does not exclude the presence of small amounts of active agents that becomes complexed over time. In embodiments having more than one active agent, at least one of those active agents can be complexed with the cyclodextrin, and at least one of the other active agents is not complexed with the cyclodextrin. In other embodiments, none of the active agents are complexed with the cyclodextrin, while in further embodiments all the active agents can be complexed with the cyclodextrin. Active agents may be complexed with cyclodextrins using conventional chemistries, and the resulting complex then added to the hydrogel. In non-complexed embodiments, the cyclodextrin and active agent(s) may be separately added to the hydrogel, either as neat compositions or in a pharmaceutically acceptable carrier or solvent.

A cyclodextrin is a macrocyclic oligosaccharide, typically a macrocycle composed of D-glucose residues linked by 1,4-β-glycosidic bonds. In some embodiments, the cyclodextrin can be either modified or unmodified. Unmodified cyclodextrins are those in which none of the hydroxyl groups have been capped or removed from the carbohydrate residues (i.e., chemically converted to a non-hydroxyl functional group). A modified cyclodextrin refers to a compound in which one or more of the hydroxyl groups in the carbohydrate residues has been converted to a different functional group, e.g., an alkoxy, an acyl, a hydroxyalkoxy, a sulfoalkoxy, or a carboxyalkoxy. The skilled person understands that modified cyclodextrins typically do not exist as a single compound, but are provided as mixtures in which 3, 2, 1, or none of the hydroxyl groups in a given carbohydrate residue have been modified. In some embodiments, cyclodextrin can be represented by the formula:

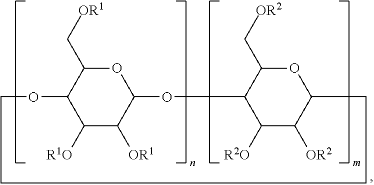

wherein n is selected from 1-8, m is selected from 0-7, and the sum of n+m is 6, 7 or 8; R¹ is in case independently selected from hydrogen, acyl, e.g., C₁₋₄acyl, alkyl, e.g., C₁₋₄alkyl, hydroxyalkyl, e.g., C₁₋₄hydroxyalkyl, carboxyalkyl, e.g., C₁₋₄carboxyalkyl, and sulfoalkyl, e.g., C₁₋₄sulfoCalkyl; and R² is in each case independently selected from hydrogen, acyl, e.g., C₁₋₄ acyl, alkyl, e.g., C₁₋₄alkyl, hydroxyalkyl, e.g., C₁₋₄hydroxyalkyl, carboxyalkyl, e.g., C₁₋₄carboxyalkyl, and sulfoalkyl, e.g., C₁₋₄sulfoalkyl.

Exemplary alkyl groups include methyl and ethyl. Exemplary acyl groups include acetate (CH₃C(═O)—) and isobutyryl ((CH₃)₂CHC(═O)—). Exemplary hydroxyalkyl groups include 2-hydroxyethyl, poly(ethylene glycol) and 2-hydroxypropyl, said hydroxy groups may be further derivatized with methyl capping groups. Exemplary carboxyalkyl groups include carboxymethyl. Exemplary sulfoalkyl groups include 4-sulfobutyl.

When n+m is 6, the cyclodextrin may be designated as an “α” cyclodextrin, when n+m is 7, the cyclodextrin may be designated as an “β” cyclodextrin, and when n+m is 8, the cyclodextrin may be designated as an “γ” cyclodextrin. An unmodified, six residue cyclodextrin can be designated as “α-cyclodextrin.” An unmodified, seven residue cyclodextrin can be designated as “β-cyclodextrin.” An unmodified, eight residue cyclodextrin can be designated as “γ-cyclodextrin.”

In further embodiments, the cyclodextrin can be composed of D-glucose units, that is the cyclodextrin has the formula:

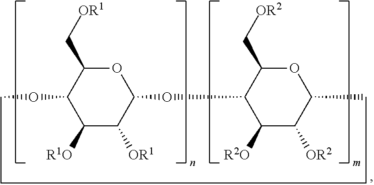

wherein n, m, R¹, and R² are as defined above.

Exemplary cyclodextrins include wherein R¹ and R² are independently selected from H and hydroxyethyl, 2-hydroxypropyl, methyl, carboxymethyl, and sulfobutyl. In some embodiments, the cyclodextrin is selected from α-cyclodextrin, β-cyclodextrin, γ-cyclodextrin, (2-hydroxypropyl)-β-cyclodextrin, (2-hydroxypropyl)-γ-cyclodextrin, methyl-α-cyclodextrin, sulfobutyl-ether-β-cyclodextrin.

The hydrogel can include a polypeptide (or a combination of different polypeptides, e.g., a first polypeptide, a second polypeptide, etc.), for instance the first polypeptide can have an average molecular weight from 15,000-150,000, from 20,000-50,000, from 20,000-30,000, from 30,000-75,000, from 40,000-60,000, from 40,000-50,000, from 50,000-150,000, from 50,000-125,000, or from 50,000-100,000. Likewise, the second (and third, fourth, etc) polypeptide can have an average molecular weight from 15,000-150,000, from 20,000-50,000, from 20,000-30,000, from 30,000-75,000, from 40,000-60,000, from 40,000-50,000, from 50,000-150,000, from 50,000-125,000, or from 50,000-100,000.

Exemplary polypeptides that may be advantageously employed in the hydrogel include those having glycine, proline, and hydroxyproline residues. For example, the polypeptide can include glycine, proline, and hydroxyproline residues in a combined number that is at least 25%, at least 30%, at least 35%, at least 40%, at least 45%, at least 55%, or at least 60%, relative to the total number of amino acid residues in the polypeptide. In certain embodiments, the polypeptide can include glycine residues in a number that from 20-40% relative to the total number of amino acid residues in the polypeptide. In some embodiments, the polypeptide can include proline residues in a number that from 7.5-22.5% relative to the total number of amino acid residues in the polypeptide. In further embodiments, the polypeptide can include hydroxyproline residues in a number that from 5-20% relative to the total number of amino acid residues in the polypeptide. In preferred embodiment, the polypeptide includes glycine residues in a number that from 20-40%, proline residues in a number that from 7.5-22.5%, and hydroxyproline residues in a number that from 5-20%, each relative to the total number of amino acid residues in the polypeptide

In further embodiments the polypeptide can contain arginine residues in a number that is from 5-20%, from 5-15%, or from 5-10% relative to the total number of amino acid residues in the polypeptide.

In certain instances, the polypeptide can include aspartic acid residues in a number that is from 3-12%, from 3-9%, or from 5-9% relative to the total number of amino acid residues in the polypeptide.

In some embodiments, the polypeptide can include glutamic acid residues in a number that is from 5-20%, from 5-15%, or from 5-10% relative to the total number of amino acid residues in the polypeptide.

In some implementations, the hydrogel includes gelatin, hyaluronic acid, or a combination thereof. In some embodiments the gelatin can be type A gelatin, type B gelatin, or a combination thereof, and can have a Bloom number from 225 to 325, from 175-225, from 100-175, or from 50-125.

In some embodiments, the hydrogel can include a crosslinked polypeptide, for example a crosslinked gelatin. In some embodiments the hydrogel can include a first polypeptide that is crosslinked, and a second polypeptide that is not crosslinked. Covalent crosslinking is a preferred type of crosslinking for the polypeptide, which may be carried out with one or more chemical crosslinking agents and/or one or more crosslinking enzymes.

Exemplary chemical crosslinking agents include aldehydes, carbodiimides, α,β-unsaturated esters, acids, and malonates, and combinations thereof. In some embodiments, the crosslinking agent includes glutaraldehyde, genipin, 1-ethyl-3-[3-dimethylaminopropyl] carbodiimide (“EDC”) or p-phenylene biscarbodiimide (“BCDI”); each of which may further include N-hydroxysuccinimide (“NHS”).

In some embodiments, the “crosslinking ratio” of a given crosslinked polypeptide can be designated by the initial ratio of crosslinker:target functional group in the peptide, rather than any specific number of crosslinked bonds in the gel. For example, a peptide crosslinked with EDC using a carbodiimide:COOH_{polypeptide} of 0.2:1 may be said to have a crosslinking ratio 1:5. As used herein, the functional group number is based on the average number of the functional groups in a given polypeptide compound.

The gels disclosed herein can have a crosslinking ratio from 10:1 to 1:10, from 5:1 to 1:5, from 10:1 to 1:1, from 10:1 to 5:1, from 7.5:1 to 2.5:1 from 5:1 to 1:1, from 2:5:1 to 1:1, from 2.5:1 to 1:2.5, from 1:1 to 1:2.5, from 1:1 to 1:5 from 1:2.5 to 1:7.5, from 1:5 to 1:10, or from 1:1 to 1:10.

In some embodiments the “degree of crosslinking” of a given crosslinked peptide can be designated by the fraction of crosslinked functional groups relative to the total number of functional group (crosslinked and uncrosslinked) in the gel. For example, a polypeptide in which 20% of all carboxylic acid groups are crosslinked and the other 80% are unmodified may be said to have an 20% degree of crosslinking. Degree of crosslinking may be determined by spectroscopic analysis (e.g., by NMR) or by chemical modification of the free carboxylic acid groups with an easily detectable functional group.

The gels disclosed herein may have a degree of crosslinking from 5-100%, from 25-100%, from 50-100%, from 75-100%, from 5-25%, from 15-35%, from 25-50%, from 30-60%, from 40-70%, from 50-75%, from 60-80%, or from 60-90%.

In some embodiments, the polypeptide may be crosslinked enzymatically, for instance using an enzyme such transglutaminase, tyrosinase, or horseradish peroxidase.

When hydrated, the hydrogels can contain from 70-99.99% by weight of water, relative to the weight of the total composition. In some embodiments, the hydrogel compositions can contain from 75-99.99%, from 80-99.99%, from 85-99.99%, from 90-99.99%, from 92.5-99.99%, from 95-99.99%, from 97.5-99.99%, from 85-98%, from 90-98%, from 92.5-98%, from 95-98%, from 97.5-98%, from 90-96%, from 92.5-96%, from 95-96%, from 92.5-97.5%, from 93-97%, or from 94-97% by weight of water, relative to the weight of the total composition.

The composition may include one or more HMG-COA inhibitor. Such agents can be referred to as statins. Exemplary HMG-COA inhibitors include cerivastatin, itavastatin, pitavastatin, simvastatin, simvastatin acid, mevastatin, 3′-hydroxy simvastatin acid, 6′-hydroxymethyl simvastatin acid, lovastatin, atorvastatin, fluvastatin, pravastatin, and rosuvastatin. The HMG-COA inhibitor may be present at a concentration, relative to the total volume water in the hydrogel, from 0.1-1,000 mM, from 0.1-750 mM, from 0.1-500 mM, from 0.1-250 mM, from 0.1-100 mM, from 0.1-75 mM, from 0.1-50 mM, from 0.1-25 mM, from 0.1-15 mM, from 0.1-10 mM, from 0.1-5 mM, from 1-100 mM, from 5-100 mM, from 10-100 mM, from 25-100 mM, from 50-100 mM, from 75-100 mM, from 5-75 mM, from 10-75 mM, from 25-75 mM, from 50-75 mM, from 5-50 mM, from 10-50 mM, or from 25-50 mM. For embodiments in which more than one HMG-COA inhibitor is present, the concentration ranges given above refer to the sum total of the HMG-COA inhibitor in the composition.

The composition may include one or more cyclodextrins in a concentration, relative to the total volume water in the hydrogel, from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM. For embodiments in which more than one cyclodextrin inhibitor is present, the concentration ranges given above refer to the sum total of the cyclodextrins in the composition. For embodiments in which a portion of one or more cyclodextrins is complexed with one or more active agents, the concentration ranges refer to the sum total of all cyclodextrins in the composition.

The compositions may include a single HMG-COA inhibitor and a single cyclodextrin. In other embodiments, the composition may include a single HMG-COA inhibitor and two cyclodextrins, wherein the cyclodextrins are not the same. In such cases, the HMG-COA can be complexed with one of the cyclodextrins, and not complexed with the other cyclodextrin. For example, the HMG-COA can be complexed with an unmodified cyclodextrin, and the composition can further include a modified cyclodextrin that is not complexed with the HMG-CoA inhibitor. In other embodiments, the HMG-COA can be complexed with a modified cyclodextrin, and the composition can further include an unmodified cyclodextrin that is not complexed with the HMG-COA inhibitor. In yet further embodiments, the HMG-COA can be complexed with a first modified cyclodextrin, and the composition can further include a second modified cyclodextrin that is not complexed with the HMG-COA inhibitor, wherein the first modified cyclodextrin and second modified cyclodextrin are not the same. By way of example, the modified cyclodextrin that is complexed with the HMG-COA inhibitor can be modified with non-ionic groups (e.g., alkyl, hydroxyalkyl, acyl) and the modified cyclodextrin that is not complexed with the HMG-COA inhibitor can be modified with ionic groups (e.g., carboxy or sulfo). In other embodiments, the modified cyclodextrin that is complexed with the HMG-COA inhibitor can be modified with alkyl groups and the modified cyclodextrin that is not complexed with the HMG-COA inhibitor can be modified with hydroxyalkyl groups.

In other embodiments, the HMG-COA inhibitor is not complexed with either cyclodextrin, and in yet further embodiments, the HMG-COA inhibitor can be complexed with both cyclodextrins, i.e., a portion of the HMG-COA inhibitor is complexed with the first cyclodextrin, and a separate portion of the HMG-COA inhibitor is complexed with the second cyclodextrin, where the first and second cyclodextrins are defined above.

The molar ratio of the HMG-COA inhibitor:cyclodextrin can be from 1:1 to 1:100, from 1:1 to 1:500, from 1:1 to 1:250, from 1:1 to 1:100, from 1:1 to 1:75, from 1:1 to 1:50, from 1:1 to 1:25, from 1:1 to 1:20, from 1:1 to 1:15, from 1:1 to 1:10, from 1:1 to 1:5, from 1:500 to 1:1,000 from 1:250 to 1:1,000, from 1:250 to 1:750, from 1:250 to 1:500, from 1:100 to 1:250, from 1:50 to 1:250, from 1:50 to 1:150, from 1:25 to 1:100, from 1:25 to 1:75, from 1:25 to 1:50, from 1:10 to 1:50, from 1:5 to 1:25, from 1:20 to 1:40, from 1:30 to 1:60, from 1:40 to 1:70, from 1:50 to 1:80, from 1:60 to 1:90, from 1:70 to 1:100, from 1:80 to 1:110, from 1:90 to 1:120, from 1:100 to 1:130. When multiple cyclodextrins are present, the ratios reflect the total amount of cyclodextrin.

The composition may include a single HMG-COA inhibitor, or the composition may include two or more HMG-COA inhibitors. For example, the composition may include a first HMG-COA inhibitor, and a second HMG-COA inhibitor. In some embodiments, the first HMG-CoA inhibitor is simvastatin, and the second HMG-CoA inhibitor is mevastatin, lovastatin, atorvastatin, or rosuvastatin. In some embodiments, the first HMG-COA inhibitor is lovastatin, and the second HMG-COA inhibitor is mevastatin, simvastatin, atorvastatin or rosuvastatin. In some embodiments, the first HMG-COA inhibitor is atorvastatin, and the second HMG-COA inhibitor is mevastatin, lovastatin, simvastatin, or rosuvastatin. In some embodiments, the first HMG-CoA inhibitor is rosuvastatin, and the second HMG-COA inhibitor is mevastatin, lovastatin, atorvastatin, or simvastatin.

The first HMG-COA inhibitor may be combined with the second HMG-COA inhibitor in a molar ratio from 5:1 to 1:5, from 2.5:1 to 1:2.5, from 1.5:1 to 1:1.5, from 5:1 to 1:1, from 5:1 to 2.5:1, from 4:1 to 2:1, from 3:1 to 1:1, from 2:1 to 1:1, from 1:1 to 5:1, from 1:2.5 to 1:5, from 1:2 to 1:4, from 1:1 to 1:3, or from 1:1 to 1:2.

In some embodiments, the first HMG-COA inhibitor is complexed with a cyclodextrin, while the second HMG-COA inhibitor is not complexed with a cyclodextrin. In other embodiments, both the first and second HMG-COA inhibitors are complexed with a cyclodextrin, and in yet further embodiments, neither the first nor the second HMG-COA inhibitor are complexed with the cyclodextrin. In certain cases, the first HMG-COA inhibitor can be complexed with a first cyclodextrin, and the second HMG-COA inhibitor can be complexed with a second cyclodextrin, wherein the first and second cyclodextrins are not the same. For example, the first cyclodextrin can be an unmodified cyclodextrin, for example a compound in which each of R¹ and R² are in each instance hydrogen, for example α-cyclodextrin, β-cyclodextrin, or γ-cyclodextrin), while the second cyclodextrin can be a modified cyclodextrin, e.g., a compound in which either of R¹ or R² is not in every instance hydrogen, for example (2-Hydroxypropyl)-β-cyclodextrin, (2-hydroxypropyl)-γ-cyclodextrin, methyl-α-cyclodextrin, or sulfobutyl-ether-β-cyclodextrin.

The molar ratio of the combined amount for both HMG-COA inhibitors:cyclodextrin can be from 1:1 to 1:100, from 1:1 to 1:500, from 1:1 to 1:250, from 1:1 to 1:100, from 1:1 to 1:75, from 1:1 to 1:50, from 1:1 to 1:25, from 1:1 to 1:20, from 1:1 to 1:15, from 1:1 to 1:10, from 1:1 to 1:5, from 1:500 to 1:1,000 from 1:250 to 1:1,000, from 1:250 to 1:750, from 1:250 to 1:500, from 1:100 to 1:250, from 1:50 to 1:250, from 1:50 to 1:150, from 1:25 to 1:100, from 1:25 to 1:75, from 1:25 to 1:50, from 1:10 to 1:50, from 1:5 to 1:25, from 1:20 to 1:40, from 1:30 to 1:60, from 1:40 to 1:70, from 1:50 to 1:80, from 1:60 to 1:90, from 1:70 to 1:100, from 1:80 to 1:110, from 1:90 to 1:120, from 1:100 to 1:130. When multiple cyclodextrins are present, the ratios reflect the total amount of cyclodextrin.

In some embodiments, the composition includes a first, second, and third HMG-COA inhibitor. In certain cases the three HMG-COA inhibitors are selected from cerivastatin, itavastatin, pitavastatin, simvastatin, mevastatin, lovastatin, atorvastatin, fluvastatin, pravastatin, and rosuvastatin, or are selected from, simvastatin, mevastatin, lovastatin, atorvastatin, fluvastatin, pravastatin, and rosuvastatin, and more preferably selected from simvastatin, mevastatin, lovastatin, atorvastatin, and rosuvastatin. For embodiments in which the composition contains a first, second, and third HMG-COA inhibitor, the inhibitors may be combined in the following ratios, relative to the total moles of HMG-COA inhibitor in the composition:

| First HMG-CoA | Second HMG-CoA | Third HMG-CoA |
|---|---|---|
| Inhibitor | Inhibitor | Inhibitor |
| 5-90% | 5-90% | 5-90% |
| 50-90% | 25-75% | 5-25% |
| 5-25% | 50-90% | 25-75% |
| 25-75% | 5-25% | 50-90% |
| 30-60% | 30-60% | 30-60% |
| 50-90% | 25-50% | 25-50% |
| 25-50% | 50-90% | 25-50% |
| 25-50% | 25-50% | 50-90% |
| 50-90% | 2-10% | 25-50% |
| 25-50% | 50-90% | 2-10% |
| 2-10% | 25-50% | 50-90% |
| 80-96% | 2-10% | 2-10% |
| 2-10% | 80-96% | 2-10% |
| 2-10% | 2-10% | 80-96% |
| 50-90% | 5-25% | 5-25% |
| 5-25% | 50-90% | 5-25% |
| 5-25% | 5-25% | 50-90% |

In some embodiments, none of the first, second, or third HMG-COA inhibitors are complexed with a cyclodextrin. In other embodiments, the first HMG-COA inhibitor is complexed with a cyclodextrin, while the second and third HMG-COA inhibitors are not complexed with a cyclodextrin. In other embodiments, both the first and second HMG-COA inhibitors are complexed with a cyclodextrin, while the third HMG-COA is not complexed with a cyclodextrin. For such embodiments, the first and second HMG-COA inhibitors may be complexed with the same cyclodextrin, or with different cyclodextrins, as defined above. In other embodiments, both the second and third HMG-COA inhibitors are complexed with a cyclodextrin, while the first HMG-COA inhibitor is not complexed with a cyclodextrin. For such embodiments, the second and third HMG-COA inhibitors may be complexed with the same cyclodextrin, or with different cyclodextrins, as defined above. In certain cases, each of the first, second, and third HMG-COA inhibitors can be complexed with a cyclodextrin, for such embodiments, all three inhibitors may be complexed with the same cyclodextrin, or different cyclodextrins can be complexed with each HMG-COA inhibitor. For example, the first HMG-CoA inhibitor can be complexed with an unfunctionalized cyclodextrin, the second HMG-COA inhibitor can be complexed with cyclodextrin that is functionalized with non-ionic groups (e.g., methyl or 2-hydroxypropyl), and the third HMG-COA inhibitor can be complexed with a cyclodextrin that is functionalized with ionic groups (e.g., carboxymethyl or sulfobutyl).

The molar ratio of all three HMG-COA inhibitors:cyclodextrin can be from 1:1 to 1:100, from 1:1 to 1:500, from 1:1 to 1:250, from 1:1 to 1:100, from 1:1 to 1:75, from 1:1 to 1:50, from 1:1 to 1:25, from 1:1 to 1:20, from 1:1 to 1:15, from 1:1 to 1:10, from 1:1 to 1:5, from 1:500 to 1:1,000 from 1:250 to 1:1,000, from 1:250 to 1:750, from 1:250 to 1:500, from 1:100 to 1:250, from 1:50 to 1:250, from 1:50 to 1:150, from 1:25 to 1:100, from 1:25 to 1:75, from 1:25 to 1:50, from 1:10 to 1:50, from 1:5 to 1:25, from 1:20 to 1:40, from 1:30 to 1:60, from 1:40 to 1:70, from 1:50 to 1:80, from 1:60 to 1:90, from 1:70 to 1:100, from 1:80 to 1:110, from 1:90 to 1:120, from 1:100 to 1:130. When multiple cyclodextrins are present, the ratios reflect the total amount of cyclodextrin.

In further embodiments, the composition can include an antibiotic, antimicrobial peptide, probiotic or postbiotic. Exemplary antibiotics include streptomycin, neomycin, kanamycin, amikacin, gentamycin, tobramycin, sisomicin, arbekacin, apramycin, netilmicin, paromomycin, spectinomycin, ciprofloxacin, levofloxacin, lomefloxacin, moxifloxacin, norfloxacin, ofloxacin, sparfloxacin, trvafloxacin, gatifloxacin, gemifloxacin, cinoxacin, nalidixic acid, azithromycin, clarithromycin, dirithromycin, erythromycin, roxithromycin, troleandomycin, telithromycin, spectinomycin, indolicidin, defensin, cecropin, magainin, vancomycin, teicoplanin, telavancin, ramoplanin, decaplanin, bleomycin, colistin (polymyxin E), colistin A (polymyxin E1), colistin B (polymyxin E2), colistin sulfate, colistimethate sodium, actinomycin, bacitracin, polymyxin B, gentamicin, gentamicin sulfate, neomycin, kanamycin, tobramycin, metronidazole, clotrimazole, secnidazole, ornidazole, tinidazole, linezolid, doxycycline, tetracycline, oxytetracycline, chlortetracycline, demeclocycline, lymecycline, meclocycline, methacycline, minocycline, rolitetracycline, tigecycline, and combinations thereof.

In some preferred embodiments, the antibiotic can be mupirocin, retapamulin, bacitracin, neomycin, polymyxin, B, sulfacetamide, mafenide, ozenoxacin, erythromycin, gentamicin, meclocycline, tetracycline, silver sulfadiazine, fusidic acid, salts thereof, and combinations thereof.

In further embodiments, the composition can include an analgesic, for example opioids, capsaicin, diclofenac, lidocaine, benzocaine, methyl salicylate, trolamine, prilocaine, pramoxine, dibucaine, phenol, tetracaine, camphor, dyclonine, menthol, and combinations thereof.

In other cases, the composition can include an anti-inflammatory agent, for example, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, and combinations thereof.

In yet further embodiments, the composition can include one or more of a growth factor, cytokine, chemokine, cluster differentiation (CD) antigen, neutrophin, hormone, enzyme, viral antigen, bacterial antigen, recombinant protein, natural protein, monoclonal antibody, polyclonal antibody, donor blood serum protein, or donor blood plasma protein. Exemplary growth factors include keratinocyte growth factor (KGF), platelet derived growth factor (PDGF), transforming growth factor-beta (TGF_{β}), interleukin, activin, albumin (ALB), alpha-1-antitrypsin (SERPINA1), alpha-2-macroglobulin (A2MG), apolipotrotein B (APOB), beta-2-glycoprotein 1 (APOH), cathelicidin antimicrobial peptide (CAMP), colony stimulating factor, ceruloplasmin (CERU), complement C3 (CO3), connective tissue growth factor (CTGF), epidermal growth factor (EGF), Epigen, erythropoietin, fibroblast growth factor (FGF), fibrinogen alpha chain (FGA), galectin, haptoglobin (HPT), hemopexin (HPX), hepatoma-derived growth factor (HDGF), hepatocyte growth factor, insulin-like growth factor binding protein (IGFBP), insulin-like growth factor, insulin, kininogen-1 (KNG1), leptin, macrophage migration inhibitory factor, melanoma inhibitory factor, myostatin, noggin, nephroblastoma overexpressed (NOV), omentin, oncostatinM, osteopontin, osteoprotogerin (OPG), periostin (POSTN)), placenta growth factor, placental lactogen, plasma protease C1 inhibitor (SERPING1), plastin-2 (LCP1), prolactin, RANK ligand, retinol binding protein, serotransferrin (TRFE), stromal cell-derived factor 1 alpha (SDF1α), stomatin (STOM), stem cell factor, transforming growth factor, vascular endothelial growth factor (VEGF), and combinations thereof.

The composition may also include metallic nanoparticles, for example gold nanoparticles, silver nanoparticles, copper nanoparticles, aluminum nanoparticles, zinc nanoparticles, and mixtures thereof.

In some preferred embodiment, the composition include a first active agent component that is one or more HMG-COA inhibitors as defined above, and a second active agent component that is one or more of an antibiotic, analgesic, growth factor, cytokine, chemokine, cluster differentiation (CD) antigen, neutrophin, hormone, enzyme, viral antigen, bacterial antigen, recombinant protein, natural protein, monoclonal antibody, polyclonal antibody, donor blood serum protein, donor blood plasma protein, metallic nanoparticle, or a combination thereof. The composition can include one or more cyclodextrins, as defined above, either complexed with a portion or all of the first active agent components.

Also disclosed herein are dressings having a backing layer having a wound-facing face and an external facing face, and a first layer comprising a composition as defined herein. The backing layer can include textile, nonwoven material, polyacrylate, polymethacrylate polyurethane, polyether urethane, polyester urethane, polyether-polyamide, or other conventional material used in medical products.

In some embodiments, the first layer includes at least one active agent, and does not include any cyclodextrin, while in other embodiments, the first layer includes at least one cyclodextrin, and does not include any active agent. The first layer may include one or more HMG-COA inhibitors and one or more cyclodextrins, which may or may not be complexed with the HMG-CoA inhibitor(s) as defined herein. For example, the first layer can include a single HMG-COA inhibitor complexed with a cyclodextrin, or the first layer can include a single HMG-CoA inhibitor and a single cyclodextrin, wherein the HMG-COA inhibitor is not complexed with the cyclodextrin. In yet further embodiments, the first layer includes a HMG-COA inhibitor complexed with a cyclodextrin, and additional cyclodextrin not complexed with an active agent. In some cases, the first layer includes at least two active agents, for example a first HMG-COA inhibitor and a second HMG-COA inhibitor, which are different from each other. One of those HMG-COA inhibitors may be complexed with a cyclodextrin, and the other HMG-CoA inhibitor is not complexed with a cyclodextrin. In other embodiments, both the first and second HMG-CoA inhibitors are complexed with a cyclodextrin (which may be the same or different as defined herein). In further embodiments, the first layer includes at least two HMG-COA inhibitors and at least one cyclodextrin, wherein neither HMG-COA inhibitor is complexed with a cyclodextrin.

The first layer may be configured with the backing layer to suit the intended application. For example, in some cases, the first layer directly contacts the wound-facing face of the backing layer, while in others there is one or more first intermediate layers disposed between the wound-facing face of the backing layer and first layer. Exemplary intermediate layers include absorbent layers (e.g., cellulose, polyurethane foam), adhesive layers, and a spacer layers, for example a layer made of an impenetrable material, but having pores to allow the transport of a composition (or components thereof) from one side to the other of the spacer layer. In some cases the spacer layer may include a textile, nonwoven material, polyacrylate, polymethacrylate polyurethane, polyether urethane, polyester urethane, polyether-polyamide, or combination of such materials.

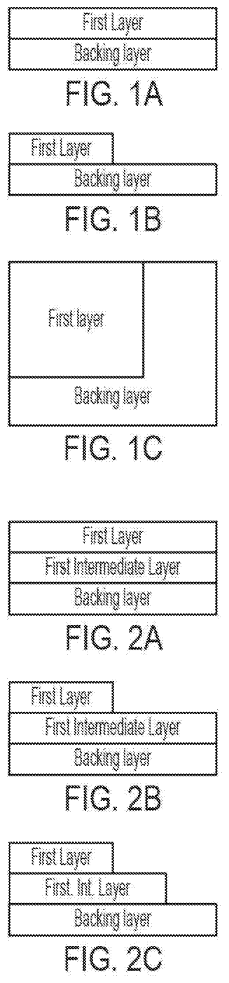

In some embodiments, the first layer directly contacts the wound-facing face of the backing layer, and is continuous with the wound-facing face of the backing layer. As used herein, a continuous layer has the same dimensions as the adjacent layer, so that the two layers completely overlap. A discontinuous layer, on the other hand, has smaller dimensions (in at least width or height) than the adjacent layer, so that the discontinuous layer does not completely overlap the adjacent layer. For example, a dressing in which the first layer is continuous with the backing layer may be represented (FIG. 1A side view representation).

In some implementations, the dressing includes a first layer that is discontinuous with the backing (FIG. 1B side view representation; FIG. 1C top view representation).

In certain implementations, the dressing includes a first intermediate layer, the first layer that may be continuous with the first intermediate layer, or may be discontinuous with the first intermediate layer, similarly, the first intermediate layer may be continuous with the backing layer or may be discontinuous with the backing layer. FIG. 2A depicts a dressing in which the first layer is continuous with the intermediate layer, and the intermediate layer is continuous with the backing layer. FIG. 2B depicts a dressing in which the first layer is discontinuous with the intermediate layer, and the intermediate layer is continuous with the backing layer. FIG. 2C depicts a dressing in which the first layer is discontinuous with the intermediate layer, and the intermediate layer is discontinuous with the backing layer.

The dressing may further have a second layer including a composition disclosed herein. In some cases, the composition in the second layer is the same as the composition in the first layer, while in other embodiments, the composition in in the second layer is different than the composition in the first layer. In some embodiments, the second layer will not include any active agent, while in other embodiments, the second layer does include an active agent and/or a cyclodextrin. For example, second layer can include at least one active agent, and does not include any cyclodextrin, or the second layer can include at least one cyclodextrin, and does not include any active agent. In further embodiments, the second layer includes at least one cyclodextrin and at least one active agent. The active agent in the second layer may be the same or different than the active agent in the first layer. For example, the first layer may include one or more HMG-CoA inhibitors and/or cyclodextrin as defined herein, and the second layer can include one of the non-HMG-CoA active agents defined herein (e.g., antibiotics, antifungals, analgesics, growth factors, etc), optionally with or without one or more cyclodextrins.

When both the first and second layers include a cyclodextrin, the cyclodextrin in the second layer can be different than the cyclodextrin in the first layer, or the cyclodextrin in the second layer is the same as the cyclodextrin in the first layer.

In some embodiments, both the first and second layers include a hydrogel as defined herein. In some embodiments, the hydrogel in the first layer is the same as the hydrogel in the second layer. In other embodiments, the hydrogel in the first layer is different in at least one aspect that the hydrogel in the second layer. For example, the crosslinked gelatin in the first layer can have a different crosslinking density than the crosslinked gelatin in the second layer, the crosslinked gelatin in the first layer is crosslinked with a different agent than the crosslinked gelatin in the second layer, or the gelatin type in the first layer is different than the gelatin type in the second layer (e.g., different bloom number or Type A vs. Type B).

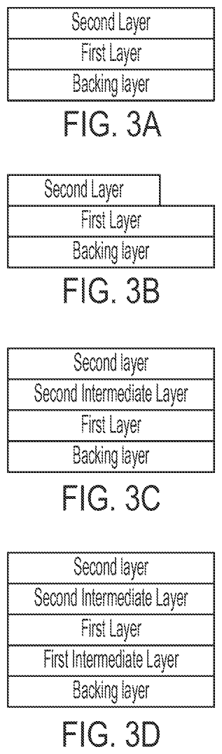

The dressing can further include a second intermediate layer between the first and second layers, wherein the second layer can be an absorbent layer, an adhesive layer, or a spacer layer. The second layer and second intermediate layer may be configured to be continuous or discontinuous with the first layer and first intermediate layer (when present). FIG. 3A depicts a dressing in which the second layer is continuous with the first layer, and the first layer is continuous with the backing layer. FIG. 3B depicts a dressing in which the second layer is discontinuous with the first layer, and the first layer is continuous with the backing layer. FIG. 3C depicts a dressing having an intermediate layer disposed between the first and second layer, in which the second layer is continuous with the intermediate layer, the intermediate layer is continuous with the first layer, and the first layer is continuous with the backing layer. FIG. 3D depicts a dressing having a second intermediate layer disposed between the first and second layer, in which the second layer is continuous with the second intermediate layer, the second intermediate layer is continuous with the first layer, a first intermediate layer disposed between the first layer and the backing layer, the first intermediate layer is continuous with the first layer and the first intermediate layer is continuous with the backing layer.

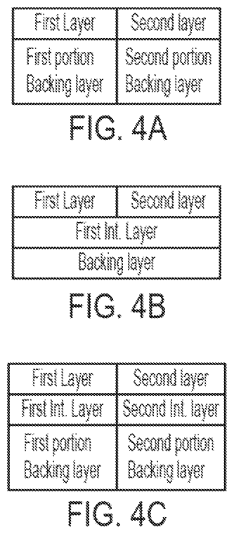

For embodiment in which the first layer is discontinuous with the backing layer or first intermediate layer, the second layer may be adjacent to the portion of the backing layer or first intermediate layer that is not adjacent to the first portion. FIG. 4A depicts a dressing having a first layer adjacent to the second layer, wherein each of the first and second layers have a backing layer that is continuous with the first and second layers. In some instances the first backing layer and the second backing layer can be the same, and in other implementations the first backing layer and the second backing layer are different. FIG. 4B depicts a dressing having a first layer adjacent to the second layer, where there is a single intermediate layer disposed between the backing layer and each of the first and second layers. FIG. 4C depicts a dressing having a first layer adjacent to the second layer, where there is a separate intermediate layer disposed between each of the first and second layers and the backing layer.

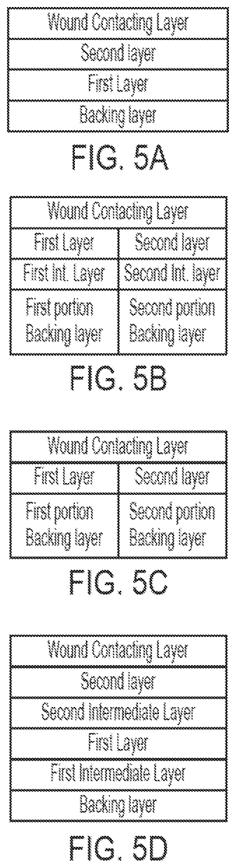

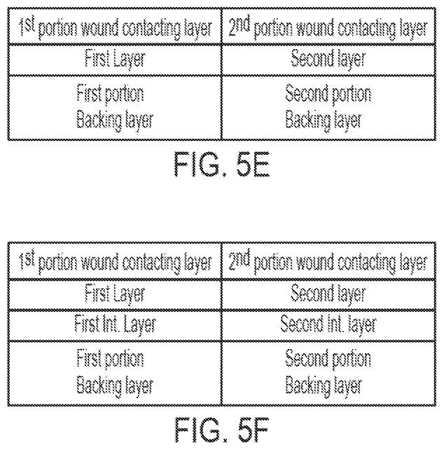

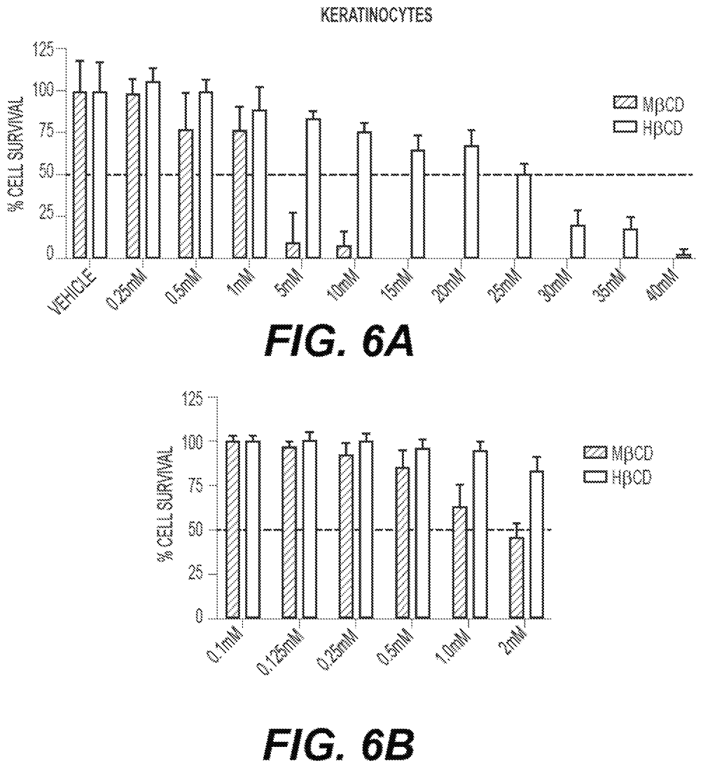

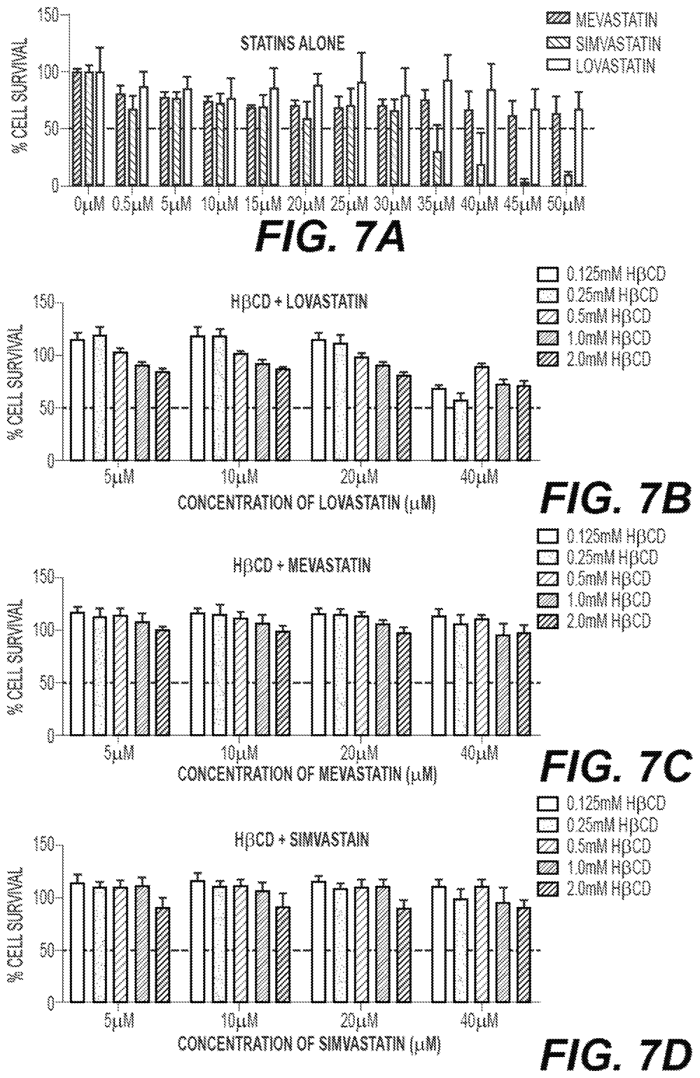

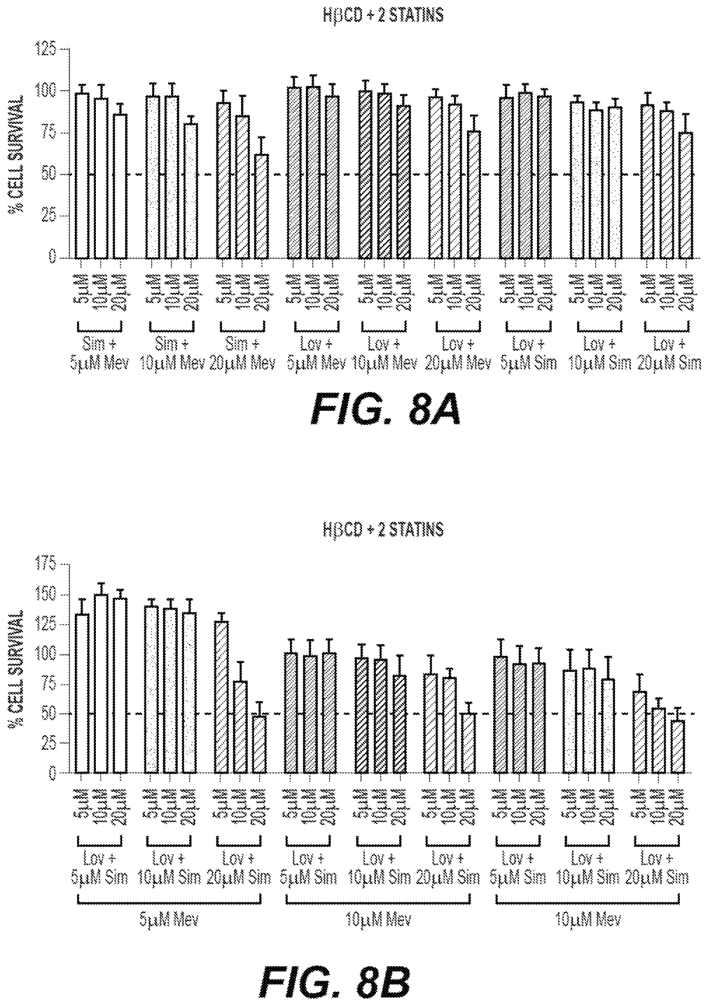

The dressing may further include one or more wound contacting layers, wherein all other layers are disposed between the wound contacting layer and the backing layer. Exemplary configurations are depicted in FIG. 5A-5F.

In some embodiments, the compositions disclosed herein may be formulated with a variety of additional excipients to give a formulation. Exemplary formulations include liniments, lotions, creams, ointments and/or pastes. Specific types of excipients that can be combined with the compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxy ethylene sorbitan [Tween 60], polyoxy ethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Compositions for rectal or vaginal administration are typically suppositories which can be prepared by mixing the conjugates with suitable non-irritating excipients such as cocoa butter, polyethylene glycol or a suppository wax which are solid at ambient temperature but liquid at body temperature and therefore melt in the rectum or vaginal cavity and release the active ingredient.

The compositions disclosed herein may be used to treat damaged tissue, and disclosed herein a methods of treating, repairing, or healing damaged tissue in a subject in need thereof. In some embodiments, the composition is directly applied to the damaged tissue. In some embodiments, the composition is applied around or adjacent to the damaged tissue. As used herein, “applied around” refers to the process of completely surrounding the damaged tissue with the composition, but not directly contacting the damaged tissue with the composition. “Applied adjacent” refers to the process of applying the composition to tissues other than the damaged tissue, wherein the one or more active agents are transported to the damaged tissue in vivo. For example, the composition may be applied in the form of a suppository. In other embodiments, the compositions may be administered orally. The composition may be applied using a dressing as described herein, having one or more active agents and layers as described herein. The composition may be applied one or more times, for instance, the composition may be applied only once to/adjacent/around the damaged, while in other embodiments, the composition is applied 2, 3, 4, 5, 6, 7, 8, 9, or 10 times.

A variety of different damaged tissues may be treated using the disclosed compositions. In some embodiments, the composition may be used to treat a wound. In certain embodiments, the damaged tissue is the result of an infection wound, mechanical wound, a thermal wound, a chemical wound, an actinic wound, a surgical would or trauma wound. The damaged tissue can be a result of decubitus ulcer, pressure ulcer, pressure sore, ulcus cruris venosum, venous ulcers, ulcus cruris arteriosum, arterial ulcer, diabetic foot, neuropathic ulcers, autoimmune disease, tumor, tropical ulcer, bacterial infection, fungal infection, necroses, or a combination thereof.

In some embodiments, the damaged tissue is a chronic wound.

In some instances, the composition can be used to a treat a wound in a subject that has diabetes, hemophilia, vitamin K deficiency, Von Willenbrand disease, or clotting factor deficiency.

In certain embodiments, the compositions can also be used to treat infected tissue. Exemplary infections that can be treated with the composition include bacterial infections, fungal infections, a parasitic infections, or viral infections, preferably bacterial infections. In certain embodiments, the composition can be used to partially rupture cellular membranes in cells infected with bacteria, fungi, or parasites. As the cell is repaired, vesicles are formed which envelop the bacteria, fungi, or parasites and expel such infectious organisms from the cell. When combined with the normal bacteriocidal, fungicidal, or aracidal (anti-parasitic) action of the active agent, the compositions can provide a multi-prong attack against infectious organisms, which can result in a superior or synergistic effect compared to active agent alone.

In certain embodiments, the compositions can be used to treat infected tissues. Exemplary infections that can be treated with the compositions include those associated with Bacillus spp., Staphylococcus spp. Streptococcus spp., Aerococcus spp., Gemella spp., Corynebacterium spp., Listeria spp., Kurthia spp., Lactobacillus spp., Erysipelothrix spp., Arachnia spp., Actinomyces spp., Propionibacterium spp., Rothia spp., Bifidobacterium spp., Clostridium spp., Eubacterium spp., Serrania spp., Klebsiella spp., Proteus spp., Enterococcus spp., Pseudomonas spp., Nocardia spp or Mycobacterium spp.

In especially preferred embodiments, the compositions disclosed herein can be used to treat the following infections: S. aureus, S. epidermidis, S. haemolyticus, S. saprophyticus, B. subtilis. B. anthracis. B. cereus, B. firmis, B. licheniformis, B. megaterium, B. pumilus, B. coagulans. B. pantothenticus, B. alvei, B. brevis. B. circubins, B. laterosporus, B. macerans, B. polymyxa, B. stearothermophilus, B. dmiringiensis, B. sphaericus, S. pyrogenes, S. pneumoniae, S. alagactiae, S. dysgalactiae, S. equisimilis, S. equi, S. zooepidemicus, S. anginosus, S. salvarius, S. milleri, S. sanguis, S. mitior, S. mutans, S. faecalis. S. faecium, S. bovis, S. equinus, S. uberus or S. avium.

In some embodiments, the composition may be used to treat a fungal infection, for example an infection with filamentous fungi or a yeast. Specific infectious organisms that may be treated include Aspergillus spp., Mucor spp., Trichtophyton spp., Cladosporium spp., Ulocladium spp., Curvularia spp., Aureobasidium spp., Candida albicans, Candida spp., Cryptococcus spp., Malessezia pachydermatis, Malessezia spp. or Trichosporon spp.

In further embodiments, the compositions disclosed herein may be used to treat infections having at least two different types of vectors, e.g., a bacterial component and a fungal component, a bacterial component and a parasitic component, a bacterial component and a viral component, a fungal component and a parasitic component, a fungal component and a viral component, or a parasitic component and a viral component.

Bacterial infections can often be accompanied by biofilm, which are complex surface attached communities of bacteria or fungi held together by a self-produced biopolymer matrix. The compositions disclosed herein may be used to treat infections characterized by the presence of biofilm.

Given the multiple modes of action provided by the disclosed compositions, the compositions and methods disclosed here may be used against hard-to-treat infections, including antibiotic resistant bacteria and fungi. Exemplary infectious organisms include carbapenem-resistant Acinetobacter, Candida auris, Clostridioides difficile, carbapenem-resistant Enterobacterales, drug-resistant Neisseria gonorrhoeae, drug-resistant Campylobacter, drug-resistant Candida, ESBL-producing enterobacterales, vancomycin-resistant Enterococci (VRE), multidrug-resistant Pseudomonas aeruginosa, drug-resistant nontyphoidal Salmonella, drug-resistant Salmonella serotype Typhi, drug-resistant Shigella, methicillin-resistant Staphylococcus aureus (MRSA), drug-resistant Streptococcus pneumoniae, drug-resistant tuberculosis, erythromycin-Resistant Group A Streptococcus, clindamycin-resistant Group B Streptococcus, azole-resistant Aspergillus fumigatus, drug-resistant Mycoplasma genitalium, and drug-resistant Bordetella pertussis.

The compositions disclosed herein may be used to treat infections expressing on the external surface of a subject, e.g., on the skin, internal infections, and infections having bother external and internal components. The composition may be applied via injection or catheter, for example to reach internally located infections. The compositions may be administered subcutaneously, intramuscularly, or intravenously, the latter being especially useful for blood-borne infections.

The compositions disclosed herein may be prepared by combining the polypeptide and crosslinker in a reaction mixture including suitable solvent. In some embodiments the solvent is water, for example at least 80%, at least 85%, at least 90%, at least 95%, or at least 99% water. The polypeptide may be provided in the solvent in a concentration from 0.1-100 mM, from 0.1-50 mM, from 0.1-25 mM, from 0.1-15 mM, from 0.1-10 mM, from 0.1-5 mM, from 0.5-10 mM, from 0.5-7.5 mM, from 0.5-5 mM, from 0.5-2.5 mM, from 1-10 mM, from 1-7.5 mM, from 1-5 mM, from 2-5 mM, from 2-8 mM, from 5-15 mM, from 10-30 mM, from 20-40 mM, from 20-60 mM, from 20-80 mM, from 30-60 mM, from 30-90 mM, from 50-100 mM, or from 50-75 mM.

The crosslinking agent may be combined with the polypeptide in various amounts depending on the intended application. Greater amounts of crosslinking agent provide more heavily crosslinked polypeptides. The polypeptide may be combined with a crosslinking in a molar ratio (crosslinker:target functional group, e.g., carbodiimide:COOH_{polypeptide} or aldehyde:NH_{2polypeptide}) from 0.1-2.0, from 0.1-1.5, from 0.1-1.25, from 0.1-1.0, from 0.2-1.0, from 0.3-1.0, from 0.4-1.0, from 0.5-1.0, from 0.6-1.0, from 0.7-1.0, from 0.8-1.0, from 0.9-1.0, from 0.1-0.5, from 0.2-0.6, from 0.25-0.75, from 0.3-0.8, from 0.5-1.25, or from 0.75-1.25. When a carbodiimide (preferably EDC) and NHS are combined to crosslink the polypeptide, the molar ratio of the carbodiimde:NHS can be from 0.05-0.5, from 0.1-0.5, from 0.2-0.5, from 0.3-0.5, from 0.1-0.3, from 0.2-0.4, or from 0.3-0.5.

The crosslinking reaction may be conducted over a period of 0.1-100 hours, of 0.2-100 hours, of 0.5-100 hours, of 0.5-2 hours, of 1-100 hours, of 1-75 hours, of 1-48 hours, of 1-36 hours, of 1-24 hours, of 1-18 hours, of 1-12 hours, of 1-6 hours, of 1-4 hours of 1-2 hours, of 2-24 hours, of 4-24 hours, of 6-24 hours, of 12-24 hours, of 18-24 hours, or 18-48 hours.

In some embodiments, the cyclodextrin(s) and/or active agent(s), including HMG-COA inhibitor may be included in the reaction mixture. In other embodiments, the cyclodextrin(s) and/or active agent(s), including HMG-COA inhibitor may be added to the already-formed crosslinked gel. In other embodiments, the active agent(s) and cyclodextrin(s) may be added to the already crosslinked gel. For example, the crosslinked gel may be prepared and optionally purified, for instance by dialysis. The gel may be lyophilized or precipitated by addition of an ant-solvent and separated from residual solvent. The dehydrated crosslinked gel may be swollen in a reconstitution solution that includes the cyclodextrin(s) and/or active agent(s), including the HMG-CoA inhibitor.

The HMG-COA inhibitor(s) may be present in the reaction mixture or reconstitution solution at a concentration from 0.1-1,000 mM, from 0.1-750 mM, from 0.1-500 mM, from 0.1-250 mM, from 0.1-100 mM, from 0.1-75 mM, from 0.1-50 mM, from 0.1-25 mM, from 0.1-15 mM, from 0.1-10 mM, from 0.1-5 mM, from 1-100 mM, from 5-100 mM, from 10-100 mM, from 25-100 mM, from 50-100 mM, from 75-100 mM, from 5-75 mM, from 10-75 mM, from 25-75 mM, from 50-75 mM, from 5-50 mM, from 10-50 mM, or from 25-50 mM.

The cyclodextrin(s) may be present in the reaction mixture or reconstitution solution at a concentration from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM.

For embodiments in which the active agent (or a portion thereof) is complexed with a cyclodextrin, the complex may be present in the reaction mixture or reconstitution solution in a concentration from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM.

### EXAMPLES

The following examples are for the purpose of illustration of the invention only and are not intended to limit the scope of the present invention in any manner whatsoever.

### Example 1: Gelatin Hydrogel Scaffold Synthesis

A 3 mM gelatin stock solution was prepared by dissolving 0.15 g/mL of Gelatin B (MW 50,000, Bloom number 225, Sigma-Aldrich, MO, USA) in a 50 mL aqueous solution at 60° C. for 1 hour at constant stirring. The solution was poured onto a 50 ml conical tube (Falcon, Corning, NY, USA) and allowed to set over night at room temperature. A 105 mM 5:1 N,N-(3-dimethylaminopropyl)-N-ethyl carbodiimide (EDC) to N-hydroxysuccinimide (NHS) stock solution was prepared by dissolving 0.0671 g EDC and 0.0081 g NHS in a 10 mL aqueous solution at room temperature. In similar fashion, gelatin B was crosslinked with EDC and NHS at 0.2, 0.6, 1, and 3 EDC/COOH_{gelatinB} molar ratios, and a constant NHS/EDC molar ratio of 0.2 at room temperature. The crosslinking reactions were performed for 1, 2, and 24 hours. EDC activates the COOH groups in gelatin, which react with the adjacent free amine groups through a nucleophilic attack, and the NHS stabilizes the intermediate reaction, enhancing the crosslinking efficiency and entrapping bioactive molecules.

After the scaffolds finished crosslinking, 3 round samples were collected with an 8-mm biopsy punch. Samples were rinsed four times with deionized (DI) water and dried inside a vacuum chamber for 30 and 60 minutes. Afterwards, they were weighed, and each placed inside a well of a 6-well plate (Corning, NY, USA) with 3 mL of DI water for 1, 2, and 20 hours. Subsequently, they were collected, dabbed with a tissue, and weighted. The degree of swelling (DS) was calculated with the following equation:

$$D S = \frac{{W}_{S} - {W}_{D}}{{W}_{D}}$$

W_{S}=Swollen weight of sample after immersion (grams)
W_{D}=Dry weight of sample after vacuum chamber (grams)

After hydrogels were allowed to crosslink for 1 hour at room temperature, 3 round samples were collected with an 8-mm biopsy punch. Samples were rinsed four times with DI water and dried inside a vacuum chamber for 30 minutes. Afterwards, they were weighed, placed inside a well of a 12-well plate with 2 mL of DI water and incubated at 37° C. for 8 days. Scaffolds were collected, dabbed with a tissue, and weighted every day over the 8-day period.

### Example 2: In Vitro Testing

Isolated primary adult human epidermal keratinocytes (HEKs) (passage <5) were cultured in 100-mm tissue-culture plates (Corning, NY, USA) with 10 mL of Keratinocyte serum-free medium (KSFM) (Gibco, MA, USA) supplemented with 5% L-glutamine, 0.2 ng/mL EGF, and 25 mg/mL bovine pituitary extract, and incubated at 37° C. and 5% CO₂. Cell media was replaced every two days and the confluency was assessed with a Leica inverted microscope. Once 70-80% confluency was reached, keratinocytes were trypsinized with 0.05% trypsin EDTA, passaged, and reseeded. Isolated primary adult human epidermal fibroblasts (HEFs) (passage <15) were cultured in 100-mm tissue culture-treated plates (Corning, NY, USA) with mL of 4.5 g/L Dulbecco's modified eagle medium (DMEM) (Gibco, MA, USA) (supplemented with 5% L-glutamine, 10% fetal bovine serum, antibiotic/antimycotic agent, and sodium pyruvate), and incubated at 37° C. and 5% CO₂. Cell media was replaced every two days and the confluency was assessed with a Leica inverted microscope. Once 70-80% confluency was reached, fibroblasts were trypsinized with 0.05% trypsin EDTA, split, and reseeded.

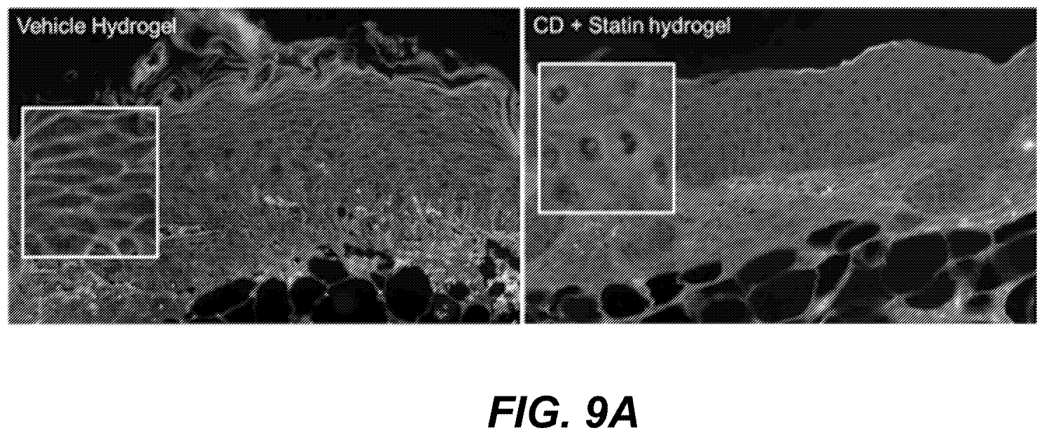

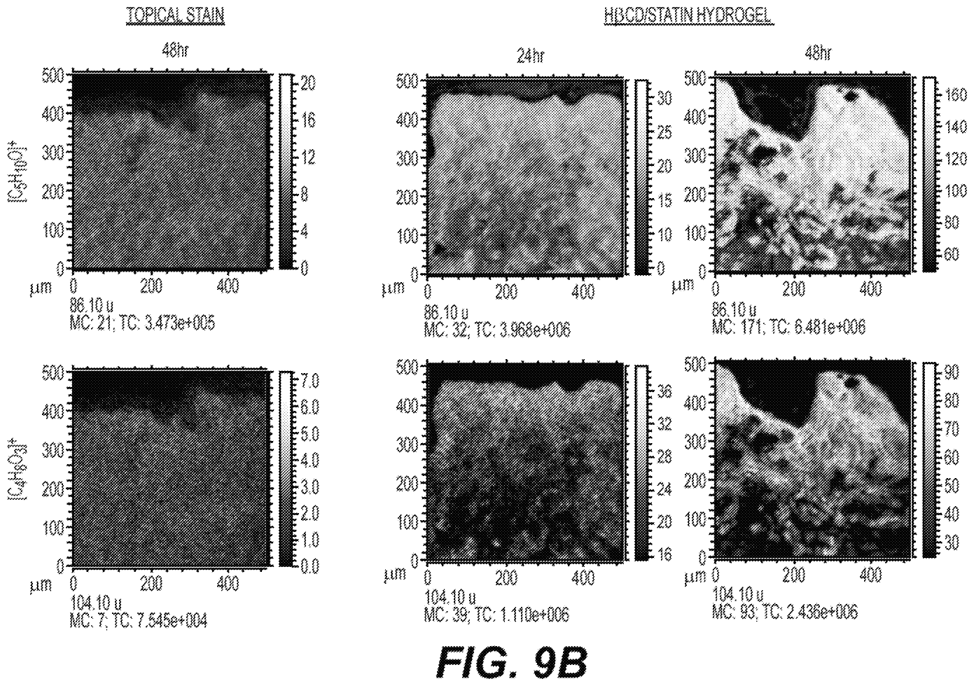

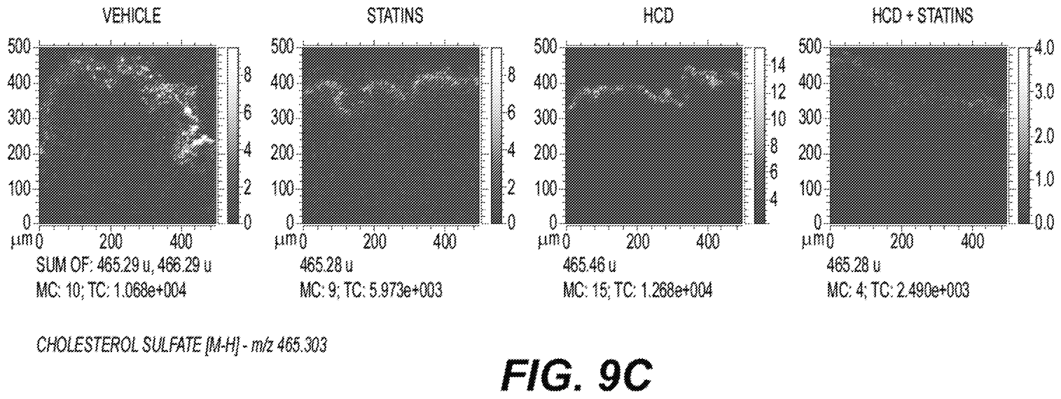

2.5 mM statin stock solutions of mevastatin (MEV), simvastatin (SIM), and lovastatin (LOV), were prepared in 100% ethanol (EtOH), and activated with sodium hydroxide. 100 mM cyclodextrin stock solutions of MβCD and HβCD were prepared in DI water. Confluent primary HEKs or HEFs were trypsinized, stained with trypan blue, and live cells counted with a hemocytometer. Subsequently, 1×10³-5×10³ cells/well were seeded in a 96-well plate (Corning, NY, USA), with 8 technical replicates per condition. After 24 hours, cell attachment was assessed, and 0-50 μM of statin were added to the cultured cells for 24 hours. Identical procedure was repeated for 0-40 mM cyclodextrin concentration treatments, and for multiple different cyclodextrins+statins combination formulations, including MβCD+MEV, HβCD+MEV, MβCD+LOV, HβCD+LOV, MβCD+SIM, HβCD+SIM, HβCD+MEV+LOV, HβCD+MEV+SIM, HβCD+LOV+SIM, and HβCD+MEV+LOV+SIM. Afterwards, treated cells were washed with phosphate-buffered saline (PBS) solution, and incubated with 100 μL/well of resazurin-based cell viability reagent (Invitrogen, MA, USA) at 37° C. for 24 hours. Lastly, absorbance at 570 nm was measured with a Benchmark Plus Microplate spectrophotometer (Bio-Rad), and the median lethal dose (LD50) was calculated for each condition. To ensure statin delivery at the skin, ex vivo human skin was topically treated with MEV+SIM alone or HβCD+MEV+SIM hydrogel for 24 or 48 hrs, after which the tissues were harvested, cryosectioned and traceable fragments of statin powder (C₅H₁₀O and C₄H₈O₃) visualized by mass spectrometry (FIG. 9b). Finally, in order to validate that the delivered statins indeed inhibit cholesterol synthesis, hydrogels containing either vehicle (growth media), MEV+SIM alone, HβCD alone, or a combination of HβCD+MEV+SIM were applied topically for 48 hrs, and levels of cholesterol sulfate determined by time of flight secondary ion mass spectrometry (FIG. 9c).

### Example 3: In Vivo Mouse Wounding

All animal care and use procedures were approved by the University of Miami Institutional Animal Care and Use Committee (IACUC #19-097). Under anesthesia, 12×7-weeks of age female db/db mice had their dorsal skin hair clipped, depilated and their fasting blood glucose level measured for 3 consecutive days, to validate hyperglycemia (>300 mg/dL was deemed as hyperglycemic). Subsequently, mice's dorsal skin was cleaned with antiseptic solution and four 5-mm round excisional wounds were made, two on each side of the dorsal midline (n=8 wounds per treatment). Dorsal wounds were treated with cylindrical hydrogel scaffolds of 8 mm×2 mm for either 7 or 16 days. Same hydrogel scaffold fabrication procedure was employed (as previously described) and with the same treatment and control formulations as the ex vivo human skin wound models. Scaffolds were fixed on top of the wounds with 14-mm square splints containing a 10 mm circular central opening. Cyanoacrylate glue was used to secure the splints just long enough for 4 non-continuous sutures to adhere the sutures to the wound and prevent wound contraction. Mice's wounded dorsal area was protected with medical dressings and elastic adhesive tape. Dorsal wounds were harvested at either at day 7 or 16, with one half of the wound fixed in 10% formalin, and paraffin-embedded, sectioned at 5-7 μm and subject to H&E staining, one quarter saved in RNAlater solution for subsequent RNA isolation, and one quarter snap frozen for protein isolation. Diabetic mice wounds' closure rate was assessed by histologically analyzing re-epithelialization and epidermal thickness through H&E staining with an Olympus VS120 microscope (Olympus, Tokyo, JPN) and Image J software (NIH, MD, USA).

Collected mouse wounds were sectioned at 5 um and used for filipin staining. Filipin was dissolved in dimethylformamide at 2.5 mg/ml, diluted to 50 ug/ml in PBS and used to incubate mouse wound sections for 30 min while protected from light. Sections were then washed twice with PBS, mounted in antifade mounting media (without DAPI) and visualized by fluorescence microscopy at 512 nm.

Amount of VEGF in snap frozen mouse wounds was analyzed using VEGF ELISA as per manufacturer's instructions. Up to 8 wounds were utilized per condition in duplicates and amount of VEGF quantified using internal standards provided by the kit.

For all internalization assays, MRSA USA300 strain was used. In vitro pathogen internalization: Primary human keratinocytes were seeded at 5×10⁴ in triplicates and allowed to adhere overnight. The following day, they were inoculated with MRSA USA300 conjugated to a pH sensitive dye (pHrodo iFL STP ester, Life Technologies) at 1×10⁶ CFU/ml for 1 hr in presence or absence of 1 mM MβCD, prior to washing with PBS. They were then either subject to live cell imaging or washed with lysostaphin for 1 hr (in order to kill any extracellular bacteria), lysed in hypotonic solution and seeded onto blood agar plates in serial dilutions in order to determine relative CFU counts. Ex vivo human skin: Human skin specimens from reduction surgery were obtained and wounded. Briefly, using a biopsy punch 4-mm acute wounds were made in the skin specimens (n=4 per treatment), and either pre-treated in presence or absence of 2 mM MβCD, followed by inoculation with 10 ul of MRSA 10⁸ CFU/ml for 24 hrs. Skin was then washed with PBS, incubated with lysostaphin for Ihr in order to kill any extracellular bacteria, lysed with hypotonic solution and subsequent lysate seeded onto blood agar plates in serial dilutions.

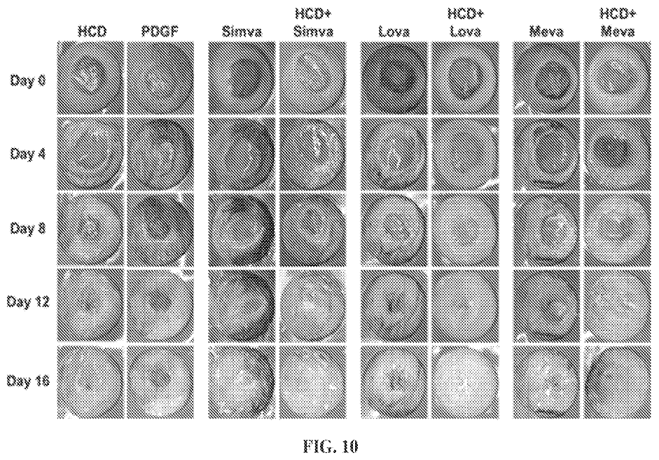

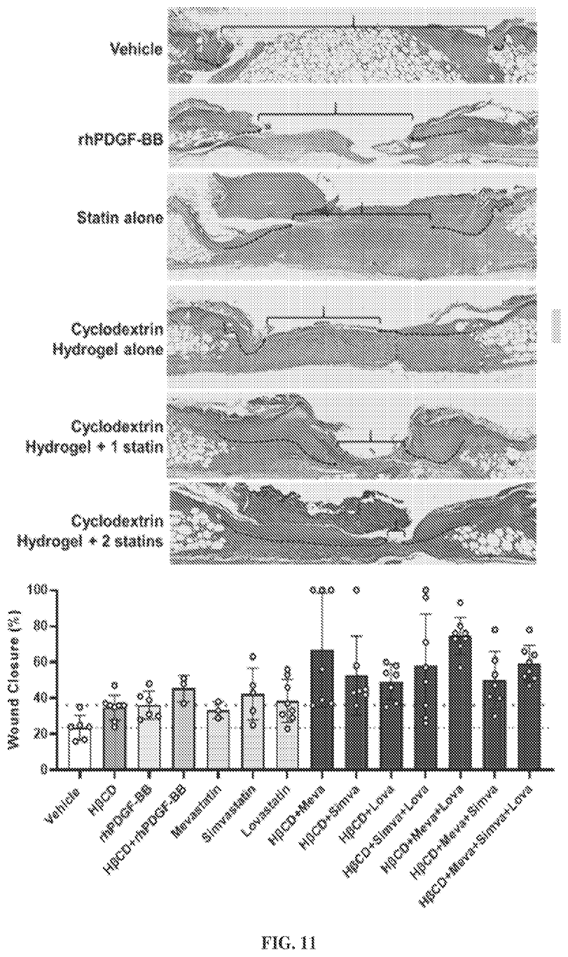

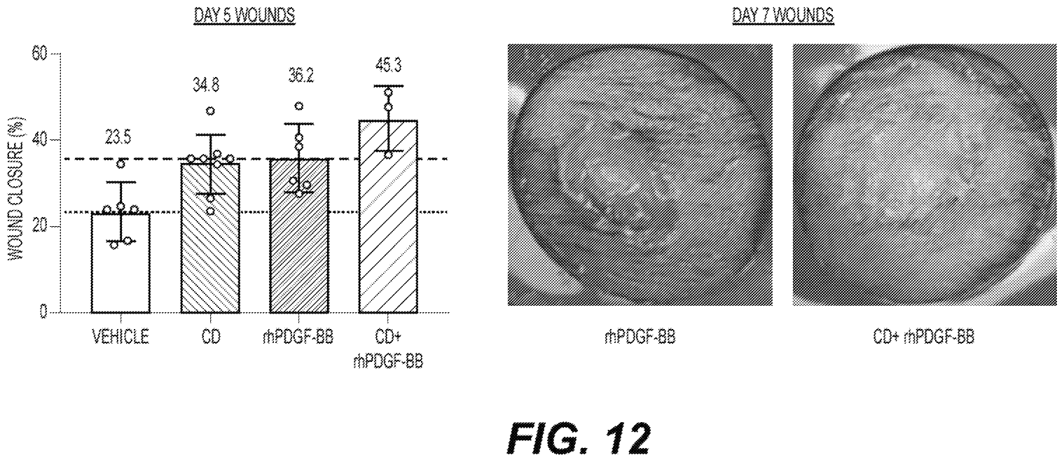

Statins alone had a very similar rate of wound closure to PDGF, incorporation of statins into HβCD containing hydrogels significantly improved rates of wound closure. Moreover, at the conclusion of the experiment, PDGF treatment was the only one with wounds that were not fully closed (FIG. 10). Histomorphometry analysis demonstrated that statins alone performed comparably to PDGF, however, their incorporation into the HβCD hydrogels exhibited a significant increase in rates of re-epithelialization (FIG. 11). Moreover, incorporation of PDGF into HβCD hydrogels resulted in an increase in relative wound closure in comparison to PDGF alone (FIG. 12). This data indicates that once growth factor receptors are released from sequestration from Cav1, subsequent growth factor administration can be more effective in eliciting a signaling response through them. As such, HβCD hydrogels as disclosed herein can be complexed to almost any other kind of growth factor to facilitate signaling through them.

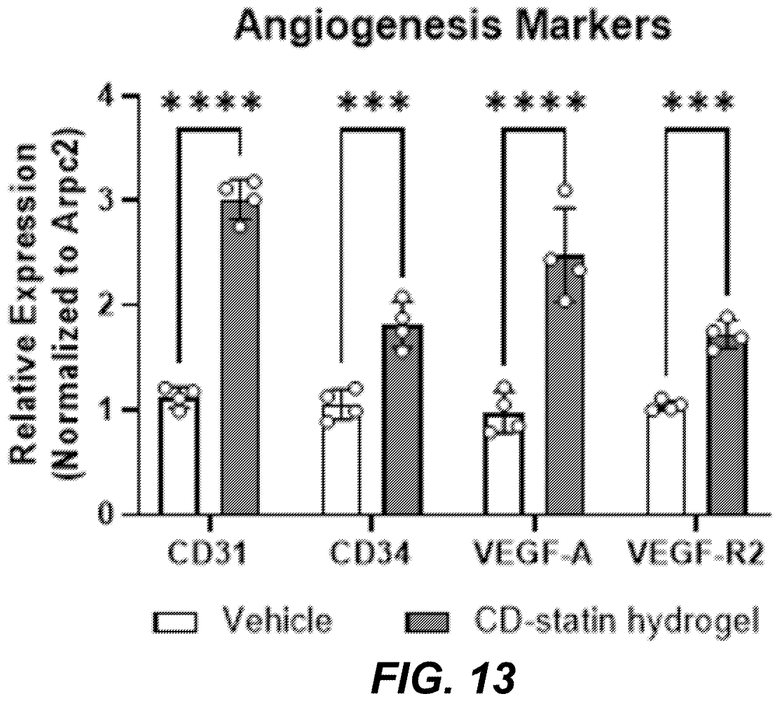

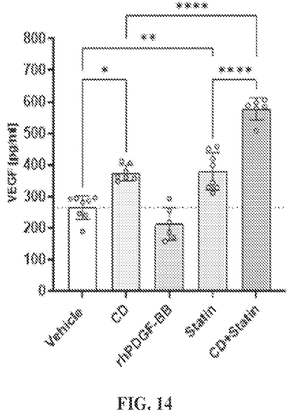

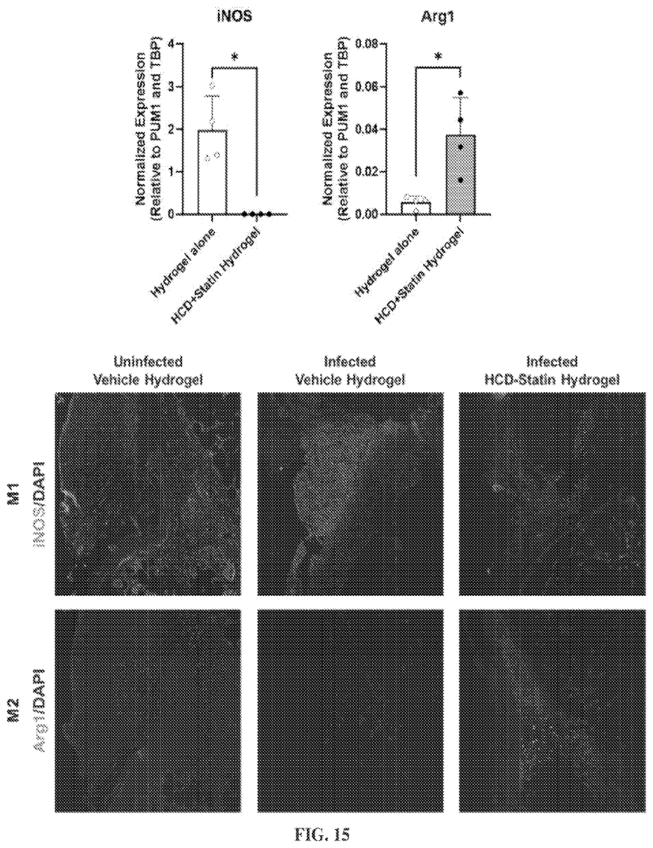

qRT-PCR was performed on some of the most common markers of angiogenesis (CD31, CD34, VEGF-A and VEGF-R2) and saw a significant increase in expression of each gene in wounds treated with our HβCD/statin hydrogel (FIG. 13). ELISA was used to measure levels of VEGF from the same wounds in order to validate that the upregulation at the mRNA level coincides with increased VEGF protein level. HβCD alone increased levels of VEGF and that any combination of statins with our HβCD hydrogel significantly increased levels of VEGF in comparison to each statin alone (FIG. 14). Moreover, HβCD-statin hydrogel promoted macrophage polarization to an M2-like phenotype, whereby it induced expression of Arg1 and reduced expression of iNOS on both mRNA and protein levels (FIG. 15).

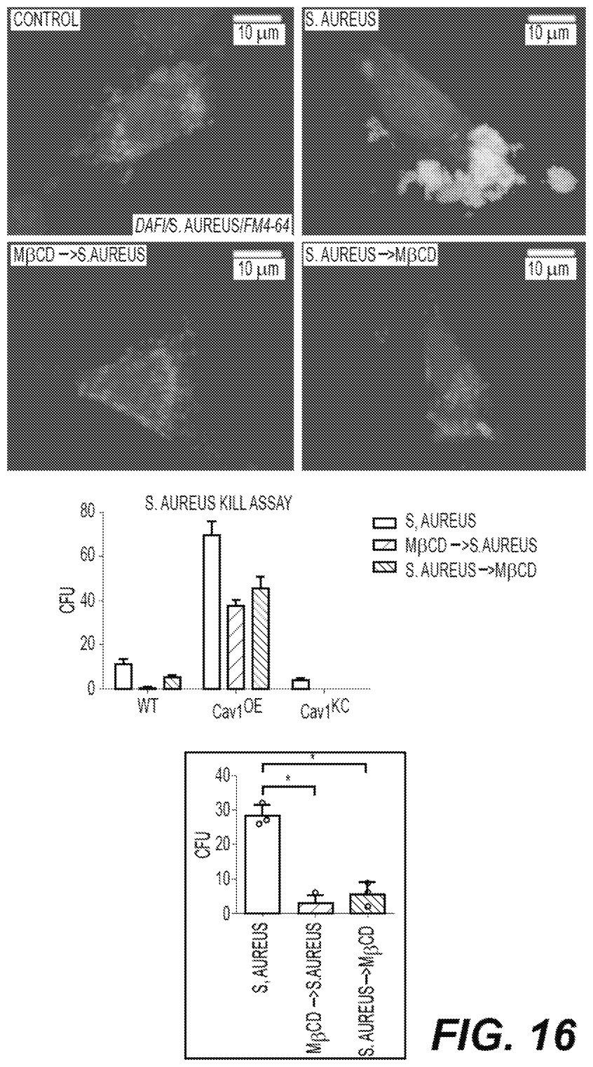

When MβCD treatment was administered after MRSA infection, it also expelled a majority of intracellular MRSA from these cells (FIG. 15). In order to confirm that the mechanism behind these observation was due to Cav1 and not any secondary effects of cyclodextrins, Cav1 overexpressing (Cav1^{OE}) and Cav1 knockout human (Cav1^{KO}) keratinocytes were treated with MRSA in presence or absence of MβCD, lysed the cells with a hypotonic buffer and then plated the resulting cell lysate on blood agar plates in order to assess levels of internalized bacteria following each treatment. MβCD treatment lowered the CFU counts and that this process is mediated by Cav1, since overexpression of Cav1 increased MRSA internalization, whereas Cav1 knockout significantly blunted it. Human skin was infected with MRSA (ex vivo) in presence/absence of MβCD and showed a similar trend in downregulation of MRSA internalization upon MβCD treatment (FIG. 16).

### FURTHER EMBODIMENTS

1. A composition comprising a cyclodextrin dispersed in a hydrogel.
2. The composition according to embodiment 1, further comprising at least one active agent.
3. The composition according to any one of embodiments 1-2, further comprising at least one active agent, wherein the active agent is complexed with the cyclodextrin.
4. The composition according to any one of embodiments 1-3, further comprising at least one active agent, wherein the active agent is separate from the cyclodextrin.
5. The composition according to any one of embodiments 1-4, comprising a mixture of at least one active agent complexed with a cyclodextrin, and at least one active agent separate from the cyclodextrin.
6. The composition according to any one of embodiments 1-5, wherein the cyclodextrin has the formula:

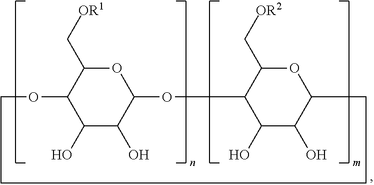

wherein n is selected from 1-8, m is selected from 0-7, and the sum of n+m is 6, 7 or 8;
R¹ is selected from hydrogen, alkyl, hydroxyalkyl, carboxyalkyl; and
R² is selected from hydrogen, alkyl, hydroxyalkyl, carboxyalkyl.
7. The composition according to any one of embodiments 1-6, wherein the cyclodextrin has the formula:

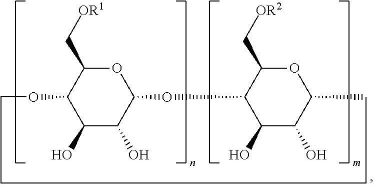

wherein n is selected from 1-8, m is selected from 0-7, and the sum of n+m is 6, 7 or 8;
R¹ is selected from hydrogen, alkyl, hydroxyalkyl, carboxyalkyl; and
R² is selected from hydrogen, alkyl, hydroxyalkyl, carboxyalkyl.
8. The composition according to any one of embodiment 1-7, wherein R¹ and R² are independently selected from H and hydroxyethyl.
9. The composition according to any one of embodiment 1-8, wherein the hydrogel comprises at least one polypeptide.
10. The composition according to any one of embodiment 1-9, wherein at least one polypeptide has an average molecular weight from 15,000-150,000, from 20,000-50,000, from 20,000-30,000, from 30,000-75,000, from 40,000-60,000, from 40,000-50,000, from 50,000-150,000, from 50,000-125,000, or from 50,000-100,000.
11. The composition according to any one of embodiment 1-10, wherein the polypeptide comprises glycine, proline, and hydroxyproline residues in a number that is at least 25%, at least 30%, at least 35%, at least 40%, at least 45%, at least 55%, or at least 60%, relative to the total number of amino acid residues in the polypeptide.
12. The composition according to any one of embodiment 1-11, wherein the polypeptide comprises glycine residues in a number that is from 20-40% relative to the total number of amino acid residues in the polypeptide.
13. The composition according to any one of embodiment 1-12, wherein the polypeptide comprises proline residues in a number that is from 7.5-22.5% relative to the total number of amino acid residues in the polypeptide.
14. The composition according to any one of embodiment 1-13, wherein the polypeptide comprises hydroxyproline residues in a number that from 5-20% relative to the total number of amino acid residues in the polypeptide.
15. The composition according to any one of embodiment 1-14, wherein the polypeptide comprises arginine residues in a number that is from 5-20%, from 5-15%, or from 5-10% relative to the total number of amino acid residues in the polypeptide.
16. The composition according to any one of embodiment 1-15, wherein the polypeptide comprises aspartic acid residues in a number that is from 3-12%, from 3-9%, or from 5-9% relative to the total number of amino acid residues in the polypeptide.
17. The composition according to any one of embodiment 1-16, wherein the polypeptide comprises glutamic acid residues in a number that is from 5-20%, from 5-15%, or from 5-10% relative to the total number of amino acid residues in the polypeptide.
18. The composition according to any one of embodiment 1-17, wherein the polypeptide comprises gelatin.
19. The composition according to any one of embodiment 1-18, wherein the polypeptide comprises gelatin, wherein the gelatin has a Bloom number from 225 to 325, from 175-225, from 100-175, or from 50-125.
20. The composition according to any one of embodiment 1-19, wherein the polypeptide comprises type A gelatin, type B gelatin, or a combination thereof.
21. The composition according to any one of embodiment 1-20, wherein the polypeptide is chemically crosslinked.
22. The composition according to any one of embodiment 1-21, wherein the polypeptide is crosslinked by one or more chemical crosslinking agents and/or one or more crosslinking enzymes.
23. The composition according to any one of embodiment 1-22, wherein the chemical crosslinking agent comprises aldehydes, carbodiimides, genipin, or a combination thereof.
24. The composition according to any one of embodiment 1-23, wherein the aldehyde comprises glutaraldehyde and the carbodiimide comprises 1-ethyl-3-[3-dimethylaminopropyl] carbodiimide (“EDC”) or p-phenylene biscarbodiimide (“BCDI”), optionally further comprising N-hydroxysuccinimide (“NHS”) 25. The composition according to any one of embodiment 1-24, wherein the crosslinking agent is EDC and NHS in a molar ratio from 0.05-0.5, from 0.1-0.5, from 0.2-0.5, from 0.3-0.5, from 0.1-0.3, from 0.2-0.4, or from 0.3-0.5.
26. The composition according to any one of embodiment 1-25, wherein the molar ratio diimide:COOH_{polypeptide} is from 0.1-2.0, from 0.1-1.5, from 0.1-1.25, from 0.1-1.0, from 0.2-1.0, from 0.3-1.0, from 0.4-1.0, from 0.5-1.0, from 0.6-1.0, from 0.7-1.0, from 0.8-1.0, from 0.9-1.0, from 0.1-0.5, from 0.2-0.6, from 0.25-0.75, from 0.3-0.8, from 0.5-1.25, or from 0.75-1.25.
27. The composition according to any one of embodiment 1-26, wherein the crosslinking enzyme comprises transglutaminase, tyrosinase, or horseradish peroxidase.
28. The composition according to any one of embodiment 1-27, wherein the crosslinked gel has a crosslinking ratio from 10:1 to 1:10, from 5:1 to 1:5, from 10:1 to 1:1, from 10:1 to 5:1, from 7.5:1 to 2.5:1 from 5:1 to 1:1, from 2:5:1 to 1:1, from 2.5:1 to 1:2.5, from 1:1 to 1:2.5, from 1:1 to 1:5 from 1:2.5 to 1:7.5, from 1:5 to 1:10, or from 1:1 to 1:10.
29. The composition according to any one of embodiment 1-28, wherein the crosslinked gel has a degree of crosslinking from 5-100%, from 25-100%, from 50-100%, from 75-100%, from 5-25%, from 15-35%, from 25-50%, from 30-60%, from 40-70%, from 50-75%, from 60-80%, or from 60-90%.
30. The composition according to any one of embodiment 1-29, wherein the hydrogel contains from 70-99.99% by weight of water, relative to the weight of the total composition.
31. The composition according to any one of embodiment 1-30, wherein the hydrogel contains from 75-99.99%, from 80-99.99%, from 85-99.99%, from 90-99.99%, from 92.5-99.99%, from 95-99.99%, from 97.5-99.99%, from 85-98%, from 90-98%, from 92.5-98%, from 95-98%, from 97.5-98%, from 90-96%, from 92.5-96%, from 95-96%, from 92.5-97.5%, from 93-97%, or from 94-97% by weight of water, relative to the weight of the total composition.
32. The composition according to any one of embodiment 1-31, wherein the active agent comprises an HMG-COA inhibitor.
33. The composition according to any one of embodiment 1-32, wherein the active agent comprises an HMG-COA inhibitor, selected from cerivastatin, itavastatin, pitavastatin, simvastatin, simvastatin acid, mevastatin, 3′-hydroxy simvastatin acid, 6′-hydroxymethyl simvastatin acid, lovastatin, atorvastatin, fluvastatin, pravastatin, rosuvastatin, or a combination thereof.
34. The composition according to any one of embodiment 1-33, wherein the active agent comprises an antibiotic.
35. The composition according to any one of embodiment 1-34, wherein the composition comprises an antibiotic at a concentration from 0.01-50 mM, from 0.05-50 mM, from 0.5-50 mM, from 1-50 mM, from 5-50 mM, from 10-50 mM, from 25-50 mM, from 0.1-10 mM, from 0.5-5 mM, from 1-15 mM, from 5-25 mM, from 5-15 mM, or from 2.5-12.5 mM.
36. The composition according to any one of embodiment 1-35, wherein the active agent comprises an antibiotic selected from streptomycin, neomycin, kanamycin, amikacin, gentamycin, tobramycin, sisomicin, arbekacin, apramycin, netilmicin, paromomycin, spectinomycin, ciprofloxacin, levofloxacin, lomefloxacin, moxifloxacin, norfloxacin, ofloxacin, sparfloxacin, trvafloxacin, gatifloxacin, gemifloxacin, cinoxacin, nalidixic acid, azithromycin, clarithromycin, dirithromycin, erythromycin, roxithromycin, troleandomycin, telithromycin, spectinomycin, indolicidin, defensin, cecropin, magainin, vancomycin, teicoplanin, telavancin, ramoplanin, decaplanin, bleomycin, colistin (polymyxin E), colistin A (polymyxin E1), colistin B (polymyxin E2), colistin sulfate, colistimethate sodium, actinomycin, bacitracin, polymyxin B, gentamicin, gentamicin sulfate, neomycin, kanamycin, tobramycin, metronidazole, clotrimazole, secnidazole, ornidazole, tinidazole, linezolid, doxycycline, tetracycline, oxytetracycline, chlortetracycline, demeclocycline, lymecycline, meclocycline, methacycline, minocycline, rolitetracycline, tigecycline, or a combination thereof.
37. The composition according to any one of embodiment 1-36, wherein the active agent comprises an antibiotic selected from mupirocin, retapamulin, bacitracin, neomycin, polymyxin, B, sulfacetamide, mafenide, ozenoxacin, erythromycin, gentamicin, meclocycline, tetracycline, silver sulfadiazine, fusidic acid, salts thereof, or a combination thereof.
38. The composition according to any one of embodiment 1-37, wherein the active agent comprises an analgesic.
39. The composition according to any one of embodiment 1-38, wherein the composition comprises an analgesic at a concentration from 0.01-50 mM, from 0.05-50 mM, from 0.5-50 mM, from 1-50 mM, from 5-50 mM, from 10-50 mM, from 25-50 mM, from 0.1-10 mM, from 0.5-5 mM, from 1-15 mM, from 5-25 mM, from 5-15 mM, or from 2.5-12.5 mM.
40. The composition according to any one of embodiment 1-39, wherein the active agent comprises an analgesic selected from an opioid, capsaicin, diclofenac, lidocaine, benzocaine, methyl salicylate, trolamine, prilocaine, pramoxine, dibucaine, phenol, tetracaine, camphor, dyclonine, menthol, or a combination thereof.
41. The composition according to any one of embodiment 1-40, wherein the active agent comprises an anti-inflammatory.
42. The composition according to any one of embodiment 1-41, wherein the composition comprises an anti-inflammatory at a concentration from 0.01-50 mM, from 0.05-50 mM, from 0.5-50 mM, from 1-50 mM, from 5-50 mM, from 10-50 mM, from 25-50 mM, from 0.1-10 mM, from 0.5-5 mM, from 1-15 mM, from 5-25 mM, from 5-15 mM, or from 2.5-12.5 mM.
43. The composition according to any one of embodiment 1-42, wherein the active agent comprises an anti-inflammatory comprising alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, or a combination thereof.
44. The composition according to any one of embodiment 1-43 wherein the active agent comprises a growth factor, cytokine, chemokine, cluster differentiation (CD) antigen, neutrophin, hormone, enzyme, viral antigen, bacterial antigen, recombinant protein, natural protein, monoclonal antibody, polyclonal antibody, donor blood serum protein, donor blood plasma protein, or a combination thereof.
45. The composition according to any one of embodiment 1-44, wherein the active agent comprises a growth factor selected from include keratinocyte growth factor (KGF), platelet derived growth factor (PDGF), transforming growth factor-beta (TGF_{β}), interleukin, activin, colony stimulating factor, connective tissue growth factor (CTGF), epidermal growth factor (EGF), Epigen, erythropoietin, fibroblast growth factor (FGF), galectin, hepatoma-derived growth factor (HDGF), hepatocyte growth factor, insulin-like growth factor binding protein (IGFBP), insulin-like growth factor, insulin, leptin, macrophage migration inhibitory factor, melanoma inhibitory factor, myostatin, noggin, nephroblastoma overexpressed (NOV), omentin, oncostatinM, osteopontin, osteoprotogerin (OPG), periostin, placenta growth factor, placental lactogen, prolactin, RANK ligand, retinol binding protein, stem cell factor, transforming growth factor, vascular endothelial growth factor (VEGF), or a combination thereof.
46. The composition according to any one of embodiment 1-45, wherein the active agent comprises metallic nanoparticles.
47. The composition according to any one of embodiment 1-46, wherein the active agent comprises metallic nanoparticles selected from gold nanoparticles, silver nanoparticles, copper nanoparticles, aluminum nanoparticles, zinc nanoparticles, and mixtures thereof.
48. The composition according to any one of embodiment 1-47, wherein the active agent comprises an anti-fungal agent.
49. The composition according to any one of embodiment 1-48, wherein the active agent comprises at least two therapeutic agents.
50. The composition according to any one of embodiment 1-49, wherein the active agent comprises a mixture comprising a first therapeutic agent that is a HMG-CoA inhibitor and second therapeutic agent that is an antibiotic, analgesic, growth factor, cytokine, chemokine, cluster differentiation (CD) antigen, neutrophin, hormone, enzyme, viral antigen, bacterial antigen, recombinant protein, natural protein, monoclonal antibody, polyclonal antibody, donor blood serum protein, donor blood plasma protein, metallic nanoparticle, or a combination thereof.
51. A dressing comprising a backing layer having a wound-facing face and an external facing face, and a first layer comprising the composition according any one of embodiment 1-XX.
52. The dressing according to embodiment 51, wherein the first layer includes at least one active agent, and does not include any cyclodextrin.
53. The dressing according to any one of embodiment 51-52, wherein the first layer includes at least one cyclodextrin, and does not include any active agent.
54. The dressing according to any one of embodiment 51-53, wherein the first layer includes at least one cyclodextrin and at least one active agent.
55. The dressing according to any one of embodiment 51-54, wherein the backing layer comprises a textile, nonwoven material, polyacrylate, polymethacrylate polyurethane, polyether urethane, polyester urethane, or polyether-polyamide.
56. The dressing according to any one of embodiment 51-55, wherein the first layer directly contacts the wound-facing face of the backing layer.
57. The dressing according to any one of embodiment 51-56, comprising one or more first intermediate layers disposed between the wound-facing face of the backing layer and first layer.
58. The dressing according to any one of embodiment 51-57, wherein the first intermediate layer comprises an absorbent layer, a spacer layer.
59. The dressing according to any one of embodiment 51-58, wherein the absorbent layer comprises cellulose, polyurethane foam.
60. The dressing according to any one of embodiment 51-59, wherein the first layer directly contacts the wound-facing face of the backing layer, and is continuous with the wound-facing face of the backing layer.
61. The dressing according to any one of embodiment 51-60, wherein the first layer directly contacts the wound-facing face of the backing layer and is discontinuous with the wound-facing face of the backing layer.
62. The dressing according to any one of embodiment 51-61, comprising a first intermediate layer disposed between the wound-facing face of the backing layer and first layer, wherein the first layer is continuous with the first intermediate layer.
63. The dressing according to any one of embodiment 51-62, comprising a first intermediate layer disposed between the wound-facing face of the backing layer and first layer, wherein the first layer is discontinuous with the first intermediate layer.
64. The dressing according to any one of embodiment 51-63, comprising a first intermediate layer disposed between the wound-facing face of the backing layer and first layer, wherein the first layer is continuous with the first intermediate layer, and wherein the first intermediate layer is continuous with the backing layer.
65. The dressing according to any one of embodiment 51-64, comprising a first intermediate layer disposed between the wound-facing face of the backing layer and first layer, wherein the first layer is continuous with the first intermediate layer, and wherein the first intermediate layer is discontinuous with the backing layer.
66. The dressing according to any one of embodiment 51-65, comprising a first intermediate layer disposed between the wound-facing face of the backing layer and first layer, wherein the first layer is discontinuous with the first intermediate layer, and wherein the first intermediate layer is continuous with the backing layer.
67. The dressing according to any one of embodiment 51-66, comprising a first intermediate layer disposed between the wound-facing face of the backing layer and first layer, wherein the first layer is discontinuous with the first intermediate layer, and wherein the first intermediate layer is discontinuous with the backing layer.
68. The dressing according to any one of embodiment 51-67, comprising a second layer comprising the composition according to any one of embodiment 1-XX.
69. The dressing according to any one of embodiment 51-68, comprising a second layer comprising the composition according to any one of embodiment 1-XX, wherein the composition in the second layer is different than the composition in the first layer.
70. The dressing according to any one of embodiment 51-69, wherein the second layer includes at least one active agent, and does not include any cyclodextrin.
71. The dressing according to any one of embodiment 51-70, wherein the second layer includes at least one cyclodextrin, and does not include any active agent.
72. The dressing according to any one of embodiment 51-71, wherein the second layer includes at least one cyclodextrin and at least one active agent.
73. The dressing according to any one of embodiment 51-72, wherein the active agent in the second layer is different than the active agent in the first layer.
74. The dressing according to any one of embodiment 51-73, wherein the active agent in the second layer is the same as the active agent in the first layer.
75. The dressing according to any one of embodiment 51-74, wherein the cyclodextrin in the second layer is different than the cyclodextrin in the first layer.
76. The dressing according to any one of embodiment 51-75, wherein the cyclodextrin in the second layer is the same as the cyclodextrin in the first layer.
77. The dressing according to any one of embodiment 51-76, wherein the hydrogel in the second layer is different than the cyclodextrin in the first layer.
78. The dressing according to any one of embodiment 51-77, wherein the hydrogel in the second layer is the same as the cyclodextrin in the first layer.
79. The dressing according to any one of embodiment 51-78, wherein the first layer and the second layer each comprise a crosslinked gelatin.
80. The dressing according to any one of embodiment 51-79, wherein the crosslinked gelatin in the first layer has a different crosslinking density than the crosslinked gelatin in the second layer.
81. The dressing according to any one of embodiment 51-80, wherein the crosslinked gelatin in the first layer is crosslinked with a different agent than the crosslinked gelatin in the second layer.
82. The dressing according to any one of embodiment 51-81, wherein the second layer directly contacts the first layer.
83. The dressing according to any one of embodiment 51-82, wherein the second layer directly contacts the first layer, and is continuous with the first layer.
84. The dressing according to any one of embodiment 51-83, wherein the second layer directly contacts the first layer, and is discontinuous with the first layer.
85. The dressing according to any one of embodiment 51-84, comprising a second intermediate layer disposed between the first layer and second layer.
86. The dressing according to any one of embodiment 51-85, wherein the second layer is continuous with the second intermediate layer, and the second intermediate layer is continuous with the first layer.
87. The dressing according to any one of embodiment 51-86, wherein the second layer is continuous with the second intermediate layer, and the second intermediate layer is discontinuous with the first layer.
88. The dressing according to any one of embodiment 51-87, wherein the second layer is discontinuous with the second intermediate layer, and the second intermediate layer is discontinuous with the first layer.
89. The dressing according to any one of embodiment 51-88, wherein the second layer is continuous with the second intermediate layer, and the second intermediate layer is discontinuous with the first layer.
90. The dressing according to any one of embodiment 51-89, wherein wound-facing face of the backing layer comprises a first portion and a second portion, the first layer directly contacting the first portion, and the second layer directly contacting the second portion.
91. The dressing according to any one of embodiment 51-90, wherein wound-facing face of the backing layer comprises:
   a first portion comprising the first layer, wherein the first intermediate layer is disposed between the first portion and first layer;
   a second portion, comprising the second layer, wherein a second intermediate layer is disposed between the second portion and second layer.
92. The dressing according to any one of embodiment 51-92, further comprising a wound contacting layer, wherein all other layers are disposed between the wound contacting layer and the backing layer.
93. The dressing according to any one of embodiment 51-93, wherein the wound contacting layer comprises a first portion and second portion, said first portion in communication with the first layer, and said second portion in communication with the second layer.
94. A method of treating damaged tissue in a subject in need thereof, comprising applying the composition according to any one of embodiment 1-50, or the dressing according to any one of embodiment 51-93 to the damaged tissue.
95. The method according to embodiment 94, wherein the damaged tissue is the result of an infection wound, mechanical wound, a thermal wound, a chemical wound, an actinic wound.
96. The method according to any one of embodiment 94-95, wherein the mechanical wound is a surgical would or trauma wound.
97. The method according to any one of embodiment 94-96, wherein the damaged tissue is a result of decubitus ulcer, pressure ulcer, pressure sore, ulcus cruris venosum, venous ulcers, ulcus cruris arteriosum, arterial ulcer, diabetic foot, neuropathic ulcers, autoimmune disease, tumor, tropical ulcer, bacterial infection, fungal infection, necroses, or a combination thereof.
98. The method according to any one of embodiment 94-97, wherein the damaged tissue is a chronic wound.
99. The method according to any one of embodiment 94-98, wherein the subject has diabetes, hemophilia, vitamin K deficiency, Von Willenbrand disease, or clotting factor deficiency.
100. A method of treating a tissue with an infection, comprising contacting the infected tissue the composition according to any one of embodiment 1-50, or the dressing according to any one of embodiment 51-93 to the damaged tissue.
101. The method according to embodiment 100, wherein the infection is a bacterial infection.
102. The method according to any one of embodiment 100-101, wherein the infection is with Bacillus spp., Staphylococcus spp. Streptococcus spp., Aerococcus spp., Gemella spp., Corynebacterium spp., Listeria spp., Kurthia spp., Lactobacillus spp., Erysipelothrix spp., Arachnia spp., Actinomyces spp., Propionibacterium spp., Rothia spp., Bifidobacterium spp., Clostridium spp., Eubacterium spp., Serratia spp., Klebsiella spp., Proteus spp., Enterococcus spp., Pseudomonas spp., Nocardia spp. Or Mycobacterium spp.
103. The method according to any one of embodiment 100-102, wherein the infection is with S. aureus, S. epidermidis, S. haemolyticus, S. saprophyticus, B. subtilis, B. anthracis, B. cereus, B. firmis, B. licheniformis, B. megateriun, B. pumilus, B. coagulans, B. pantothenticus, B. alvei. B. brevis, B. circubins, B. laterosporus, B. macerans, B. polymyxa, B. stearothermophilus, B. thuringiensis, B. sphaericus, S. pyrogenes, S. pneumomiae, S. alagactiae, S. dysgalactiae, S. equisimilis, S. equi, S. zooepidemicus, S. anginosus, S. salwarius, S. milleri, S. sanguis, S. mifior, S. mutans, S. faecalis, S. faecium. S. bovis, S. equinus, S. uberus or S. avium.
104. The method according to embodiment 100, wherein the infection in a fungal infection.
105. The method according to any one of embodiment 100 or 104, wherein the infection is with filamentous fungi or a yeast.
106 The method according to any one of embodiment 100 or 104-105, wherein the infection is with Aspergillus spp., Mucor spp., Trichtophyton spp., Cladosporium spp., Ulocladium spp., Curvularia spp., Aureobasidium spp., Candida albicans, Candida spp., Cryptococcus spp., Malessezia pachydermatis, Malessezia spp. or Trichosporon spp.
107. The method according to any one of embodiment 100-106, wherein the infection comprises a biofilm.
108. The method according to any one of embodiment 100-107, wherein the infection comprises a bacterial infection and fungal infection.
109. A method for making the composition according to any one of embodiment 1-50, comprising combining the polypeptide and crosslinker in a reaction mixture.
110. The method according to embodiment 109, wherein the reaction mixture comprises water.
111. The method according to any one of embodiment 109-110, wherein the reaction mixture comprises water in an amount of at least 80%, at least 85%, at least 90%, at least 95%, or at least 99% water, relative to the total weight of the composition.
112. The method according to any one of embodiment 109-111, wherein the polypeptide is present in the reaction mixture at a concentration from 0.1-100 mM, from 0.1-50 mM, from 0.1-25 mM, from 0.1-15 mM, from 0.1-10 mM, from 0.1-5 mM, from 0.5-10 mM, from 0.5-7.5 mM, from 0.5-5 mM, from 0.5-2.5 mM, from 1-10 mM, from 1-7.5 mM, from 1-5 mM, from 2-5 mM, from 2-8 mM, from 5-15 mM, from 10-30 mM, from 20-40 mM, from 20-60 mM, from 20-80 mM, from 30-60 mM, from 30-90 mM, from 50-100 mM, or from 50-75 mM.
113. The method according to any one of embodiment 109-112, the molar ratio (crosslinker:target functional group) is from 0.1-2.0, from 0.1-1.5, from 0.1-1.25, from 0.1-1.0, from 0.2-1.0, from 0.3-1.0, from 0.4-1.0, from 0.5-1.0, from 0.6-1.0, from 0.7-1.0, from 0.8-1.0, from 0.9-1.0, from 0.1-0.5, from 0.2-0.6, from 0.25-0.75, from 0.3-0.8, from 0.5-1.25, or from 0.75-1.25.
114. The method according to any one of embodiment 109-113, wherein the reaction mixture comprises one or more chemical crosslinking agents and/or one or more crosslinking enzymes.
115. The method according to any one of embodiment 109-114, wherein crosslinking agent comprises aldehydes, carbodiimides, genipin, or a combination thereof.
116. The method according to any one of embodiment 109-115, wherein crosslinker comprises glutaraldehyde, 1-ethyl-3-[3-dimethylaminopropyl] carbodiimide (“EDC”) or p-phenylene biscarbodiimide (“BCDI”), optionally further comprising N-hydroxysuccinimide (“NHS”).
117. The method according to any one of embodiment 109-116, wherein the crosslinking agent is EDC and NHS in a molar ratio from 0.05-0.5, from 0.1-0.5, from 0.2-0.5, from 0.3-0.5, from 0.1-0.3, from 0.2-0.4, or from 0.3-0.5.
118. The method according to any one of embodiment 109-117, where the crosslinking reaction is conducted over a period of 0.1-100 hours, of 0.2-100 hours, of 0.5-100 hours, of 0.5-2 hours, of 1-100 hours, of 1-75 hours, of 1-48 hours, of 1-36 hours, of 1-24 hours, of 1-18 hours, of 1-12 hours, of 1-6 hours, of 1-4 hours of 1-2 hours, of 2-24 hours, of 4-24 hours, of 6-24 hours, of 12-24 hours, of 18-24 hours, or 18-48 hours.
119. The method according to any one of embodiment 109-118, where the crosslinking reaction is conducted at a temperature from 15-40° C., from 15-35° C., from 15-30° C., from 20-40° C., from 20-35° C., from 20-30° C., from 30-40° C., or from 35-40° C.,
120. The method according to any one of embodiment 109-119, wherein the reaction mixture further comprises the cyclodextrin(s) and/or active agent(s), including HMG-CoA inhibitor.
121. The method according to any one of embodiment 109-120, wherein the HMG-COA inhibitor(s) is be present in the reaction mixture at a concentration from 0.1-1,000 mM, from 0.1-750 mM, from 0.1-500 mM, from 0.1-250 mM, from 0.1-100 mM, from 0.1-75 mM, from 0.1-50 mM, from 0.1-25 mM, from 0.1-15 mM, from 0.1-10 mM, from 0.1-5 mM, from 1-100 mM, from 5-100 mM, from 10-100 mM, from 25-100 mM, from 50-100 mM, from 75-100 mM, from 5-75 mM, from 10-75 mM, from 25-75 mM, from 50-75 mM, from 5-50 mM, from 10-50 mM, or from 25-50 mM.
122. The method according to any one of embodiment 109-121, wherein the cyclodextrin is present in the reaction mixture at a concentration from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM.
123. The method according to any one of embodiment 109-122, wherein the active agent (or a portion thereof) is complexed with a cyclodextrin is present in the reaction mixture at a concentration from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM.
124. The method according to any one of embodiment 109, where the reaction mixture does not include any cyclodextrin(s) and/or active agent.
125. The method according to any one of embodiment 109-124, wherein the crosslinked gel is dehydrated subsequent to the crosslinking reaction.
126. The method according to any one of embodiment 109-125, wherein the crosslinked gel is dehydrated by lyophilization or anti-solvent precipitation.
127. The method according to any one of embodiment 125-126, wherein the dehydrated crosslinked gel is swollen in a reconstitution solution that includes the cyclodextrin(s) and/or active agent(s), including HMG-COA inhibitor.
128. The method according to embodiment 127, wherein the HMG-COA inhibitor(s) is be present in the reconstitution solution at a concentration from 0.1-1,000 mM, from 0.1-750 mM, from 0.1-500 mM, from 0.1-250 mM, from 0.1-100 mM, from 0.1-75 mM, from 0.1-50 mM, from 0.1-25 mM, from 0.1-15 mM, from 0.1-10 mM, from 0.1-5 mM, from 1-100 mM, from 5-100 mM, from 10-100 mM, from 25-100 mM, from 50-100 mM, from 75-100 mM, from 5-75 mM, from 10-75 mM, from 25-75 mM, from 50-75 mM, from 5-50 mM, from 10-50 mM, or from 25-50 mM.
129. The method according to any one of embodiment 127-128, wherein the cyclodextrin is present in the reconstitution solution at a concentration from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM.
130. The method according to any one of embodiment 127-129, wherein the active agent (or a portion thereof) is complexed with a cyclodextrin is present in the reconstitution solution at a concentration from 0.1-50 mM, from 0.1-40 mM, from 0.1-30 mM, from 0.1-20 mM, from 0.1-10 mM, from 0.1-7.5 mM, from 0.1-5 mM, from 0.1-2.5 mM, from 0.1-2.0 mM, from 0.1-1.5 mM, from 0.1-1 mM, from 0.1-0.75 mM, from 0.1-0.5 mM, from 0.1-0.25 mM, from 0.25-5 mM, from 0.25-4 mM, from 0.25-3 mM, from 0.25-2 mM, from 0.25-1.5 mM, from 0.25-1.25 mM, from 0.25-1 mM, from 0.25-0.75 mM, from 0.25-0.5 mM, from 0.5-5 mM, from 0.5-2.5 mM, from 0.5-1.5 mM, from 0.5-1.0 mM, from 0.75-5 mM, from 0.75-2.5 mM, from 0.75-2 mM, from 0.75-1.5 mM, or from 0.75-1.25 mM.
131. A hydrogel composition prepared by a process according to any one of embodiment 109-130.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1-132. (canceled)

133. A composition comprising an HMG-CoA inhibitor and cyclodextrin dispersed in a hydrogel, wherein the hydrogel comprises water and a polypeptide.

134. The composition of claim 133, wherein the cyclodextrin has the formula: 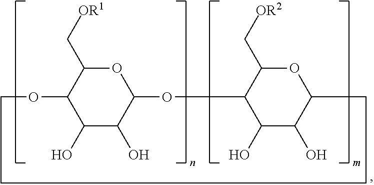
wherein n is selected from 1-8, m is selected from 0-7, and the sum of n+m is 6, 7 or 8;
R¹ is selected from hydrogen, alkyl, hydroxyalkyl, carboxyalkyl; and
R² is selected from hydrogen, alkyl, hydroxyalkyl, carboxyalkyl.

135. The composition of claim 133, wherein the cyclodextrin is α-cyclodextrin, β-cyclodextrin, γ-cyclodextrin, (2-hydroxypropyl)-β-cyclodextrin, (2-hydroxypropyl)-γ-cyclodextrin, methyl-α-cyclodextrin, sulfobutyl-ether-β-cyclodextrin, or a combination thereof.

136. The composition of claim 133, wherein the hydrogel comprises a crosslinked polypeptide.

137. The composition of claim 133, wherein the hydrogel comprises gelatin.

138. The composition of claim 133, wherein the HMG-CoA inhibitor comprises cerivastatin, itavastatin, pitavastatin, simvastatin, simvastatin acid, mevastatin, 3′-hydroxy simvastatin acid, 6′-hydroxymethyl simvastatin acid, lovastatin, atorvastatin, fluvastatin, pravastatin, rosuvastatin, or a combination thereof.

139. The composition of claim 133, further comprising an additional active agent.

140. The composition of claim 139, wherein the additional active agent comprises an antibiotic, analgesic, anti-inflammatory, growth factor, metallic nanoparticles, anti-fungal agent, or a combination thereof.

141. The composition of claim 133, wherein the HMG-CoA inhibitor comprises mevastatin, simvastatin, lovastatin, or a combination thereof.

142. The composition of claim 133, wherein the composition comprises two or three different HMG-CoA inhibitors.

143. The composition of claim 133, wherein the cyclodextrin is present at a concentration, relative to the volume of water in the hydrogel, from 0.1-10 mM.

144. The composition of claim 133, wherein the HMG-CoA inhibitor is present in amount (molar ratio HMG-CoA inhibitor:cyclodextrin) from 1:1 to 1:250.

145. The composition of claim 133, wherein the HMG-CoA inhibitor is not complexed with the cyclodextrin.

146. The composition of claim 142, wherein none of the HMG-CoA inhibitors are complexed with the cyclodextrin.

147. A method of treating damaged tissue in a subject in need thereof, comprising applying the composition of claim 133 to the damaged tissue.

148. The method of claim 147, wherein the damaged tissue is the result of an infection wound, mechanical wound, a thermal wound, a chemical wound, an actinic wound, or a combination thereof.

149. A method of treating a tissue with an infection, comprising contacting the infected tissue with the composition according to claim 133.

150. The method of claim 149, wherein the infection is a bacterial infection, fungal infection, or a combination thereof.

151. A dressing comprising a backing layer having a wound-facing face and an external facing face, and a first layer comprising the composition according to claim 133.

152. A method of making the composition of claim 133, comprising combining a polypeptide and a crosslinker in a reaction mixture, wherein the reaction mixture further includes a cyclodextrin and HMG-CoA inhibitor.
